(19) United States
(12) Patent Application Publication
(10) Pub. No.: US 2025/0360297 A1
(43) Pub. Date: Nov. 27, 2025

(54) VASCULAR ACCESS REVERSING DEVICE AND METHOD FOR TREATING ENDOVASCULAR INFARCTION

(71) Applicant: Joseph A. Ramírez-Cárdenas, Yuma, AZ (US)

(72) Inventors: Joseph A. RAMÍREZ-CÁRDENAS, Yuma, AZ (US); James P. HOPE, Nashville, TN (US); Kimberly A. LEON, Old Hickory, TN (US); Michael A. KAMM, Thompsons Station, TN (US)

(21) Appl. No.: 19/295,275

(22) Filed: Aug. 8, 2025

Related U.S. Application Data
(63) Continuation-in-part of application No. 17/936,243, filed on Sep. 28, 2022.
(60) Provisional application No. 63/261,768, filed on Sep. 28, 2021.
(60) Provisional application No. 63/681,367, filed on Aug. 9, 2024.

(51) Int. Cl. A61M 29/00; A61M 25/09; A61M 39/06

(52) U.S. Cl. CPC A61M 29/00; A61M 25/09; A61M 39/06

(57) ABSTRACT

An apparatus for providing access to a patient's vasculature according to one aspect of the present invention comprises a dilator having a distal tip, a proximal end and a main body extending therebetween. The dilator includes a first lumen extending from the proximal end to the distal tip and a second lumen extending in parallel to the first lumen between the proximal end and a side port provided in the main body spaced proximal to the distal tip by a distance. The apparatus further comprises a first hemostasis valve in fluid communication with the first lumen at the proximal end of the dilator and a second hemostasis valve in fluid communication with the second lumen at the proximal end of the dilator. A retrograde guidewire is provided positionable within the first lumen of the dilator for advancement therein from the proximal end toward the distal tip and adapted to project therefrom by a first preselected distance. A deflection microcatheter is also provided positionable within the second lumen for advancement therein from the proximal end toward the side port and adapted to project therefrom by a second preselected distance and an antegrade guidewire positionable within a lumen of the deflection microcatheter for advancement therein from the proximal end toward the side port and adapted to project therefrom by a third preselected distance.

### CROSS REFERENCE TO RELATED APPLICATIONS

This application claims priority to U.S. provisional patent application 63/681,367 filed on Aug. 9, 2024, currently pending, and is a continuation-in-part application claiming priority to U.S. patent application Ser. No. 17/936,243 filed on Sep. 28, 2022, currently pending, which claims priority to U.S. provisional patent application 63/261,768 filed on Sep. 28, 2021, now abandoned, the entire contents of each of the foregoing being hereby incorporated hereinto in their entireties as if fully rewritten herein.

### BACKGROUND

The present disclosure relates generally to apparatuses and methods for providing access to the vasculature of a patient for the purpose of treating, intervening and/or diagnosing certain vascular conditions, such as, for example, peripheral vascular disease. More specifically, the present disclosure relates to apparatuses and methods for providing access to the vasculature of a patient wherein an apparatus is provided that facilitates access to the patient's vasculature in the antegrade direction from a generally-retrograde-oriented access point.

The diagnosis and treatment of cardiovascular and endovascular disease has grown significantly in terms of sophistication, routineness and diversity. Interventional procedures involving items such as stents and balloons are virtually commonplace in many healthcare practices. One issue associated with nearly all vascular procedures relates to how to provide for optimal access to a patient's vasculature, taking into consideration such things as a patient's unique physiology, the severity and urgency of the patient's condition, the location of the offending anatomical anomaly or injury, the desired access point on the patient's body, the devices required by the procedure, and the physician's experience, expertise and preferences.

All vascular procedures begin by diagnosing the patient's condition. Peripheral artery disease, for example, is a common condition affecting blood flow to the patient's extremities, most commonly, the patient's lower legs. It is also common for peripheral artery disease to be diagnosed in a patient's arms, neck or kidneys. It is believed that risk factors for peripheral artery disease include diabetes, high blood pressure, kidney problems and high blood cholesterol. Once a diagnosis of peripheral artery disease is made, a treatment plan is prepared which may include revascularization, which is to say, to restore blood flow through the affected artery or arteries. For example, procedures such as angioplasty, atherectomy, vascular bypass, thrombolysis and thrombectomy, just to name a few, might be used to revascularize a patient. Any of these procedures, for example, require that treatment devices be delivered, via an access site into the patient's vasculature (which may be either an artery or vein), through the patient's vasculature and ultimately to the treatment location, which in some cases might be in a difficult-to-reach location of the patient's vasculature.

In the case of peripheral artery disease, the treatment location is oftentimes, for example, in the lower portion of a patient's leg, and possibly as low as the patient's ankle or into the patient's foot. The treatment condition is commonly diagnosed as severe blockages of the patient's common femoral artery due to the buildup of plaque therein. This blockage, referred-to as stenosis, must either be pushed toward the arterial sidewall (a procedure known as angioplasty) or removed from the patient's body altogether (a procedure known as atherectomy). Either procedure requires that catheters and other devices be advanced through the patient's vasculature via the access site, which in this example, typically is obtained through a needle insertion made into the patient's common femoral artery. After the needle provides access to the patient's common femoral artery, a short introducer sheath is inserted into the patient's common femoral artery over the needle, which is then removed. Procedural catheters and other devices, then, are introduced into the patient's vasculature via the introducer sheath.

As noted above in regard to this typical scenario, the access location is in the common femoral artery, also referred-to as the superficial femoral artery, which branches distally from the common femoral artery and travels “down” the patient's leg in the same direction as arterial blood flow. Because the treatment location is “below” the access site (i.e., the common femoral artery), it would be ideal, then, when gaining access to the patient's vasculature at the common femoral artery, for the access devices (including the needle and the access sheath) to be pointing “downward” (i.e., antegrade to the normal flow of blood) toward the superficial femoral artery. However, best practice for gaining access to arteries, in general, is in the retrograde direction (i.e., opposite the flow of blood). Since the access site (i.e., the common femoral artery, which is located, generally, at the patient's thigh) is “above” the treatment location, though, retrograde access at the common femoral artery would result in the introducer sheath (and the devices entering the patient's vasculature therethrough) pointing in the wrong direction.

For this reason, many procedures present a condition that requires the physician to make retrograde access to the patient's vasculature via the common femoral artery of the leg opposite to the leg in which the treatment location is presented. In this case, the introducer sheath, as well as any guidewires, catheters or other medical devices that need to be advanced through the patient's vasculature from the access site to the treatment location for the purpose of providing treatment must traverse the patient's vasculature by first being advanced retrograde through the common femoral artery of the patient's leg opposite the leg in which the treatment location is presented, then up and over the patient's iliac bifurcation, and then down the iliac artery, common femoral artery and superficial femoral artery of the leg in which the treatment location is presented.

This approach is referred-to by many as going “over the horn” and is undesirable for many reasons. First, it takes longer, thereby extending the patient's discomfort and increasing the chance that the patient's condition becomes more severe, which might include amputation due to extended periods of low or no blood flow to the extremities, or worse, it might even be fatal. Next, longer and more expensive catheters and other medical devices are necessary. Finally, the physician's orientation (while providing treatment) is turned “upside down” (i.e., for example, if the physician intends for the distal tip of the catheter to be rotated to the right, the physician must, in fact, rotate the proximal end of the device to the left). For at least these reasons, it is desirable to provide an apparatus and a method of using such apparatus that, in the most basic of terms, facilitates procedures whereby needles, access sheaths, catheters and other devices can be introduced into a patient's vasculature in a generally-retrograde direction but then advanced further through the patient's vasculature in an antegrade direction.

### SUMMARY OF THE DISCLOSURE

An apparatus and method are provided according to certain aspects of the present invention whereby antegrade advancement of a medical device, such as a guidewire, catheter, or the like, is facilitated through a patient's vasculature into which retrograde access to an artery thereof has been made and through which the medical device is introduced. According to certain aspects of the present invention, an apparatus is provided comprising a dilator having a distal tip, a proximal end and a main body extending therebetween. The dilator includes a first lumen extending from the proximal end to the distal tip and a second lumen extending in parallel to the first lumen between the proximal end and a side port provided in the main body spaced proximal to the distal tip by a distance. The apparatus further comprises a first hemostasis valve in fluid communication with the first lumen at the proximal end of the dilator and a second hemostasis valve in fluid communication with the second lumen at the proximal end of the dilator. A retrograde guidewire is provided positionable within the first lumen of the dilator for advancement therein from the proximal end toward the distal tip and adapted to project therefrom by a first preselected distance. A deflection microcatheter is also provided positionable within the second lumen for advancement therein from the proximal end toward the side port and adapted to project therefrom by a second preselected distance and an antegrade guidewire positionable within a lumen of the deflection microcatheter for advancement therein from the proximal end toward the side port and adapted to project therefrom by a third preselected distance.

According to one aspect of the present invention, there is provided as apparatus as described above, wherein the first and second hemostasis valves are connected to the proximal end of the dilator by a Y-connector such that the first hemostasis valve is in fluid communication with the first lumen of the dilator and the second hemostasis valve is in fluid communication with the second lumen of the dilator.

According to another aspect of the present invention, there is provided an apparatus as described above, wherein the first hemostasis valve is connected to a retrograde arm of the Y-connector and wherein the second hemostasis valve is connected to an antegrade arm of the Y-connector.

According to yet another aspect of the present invention, there is provided an apparatus as described above, wherein the first hemostasis valve is connected to the retrograde arm of the Y-connector by a first hemostasis valve connector and wherein the second hemostasis valve is connected to the antegrade arm of the Y-connector by a second hemostasis valve connector.

According to still yet another aspect of the present invention, there is provided an apparatus as described above, wherein the first hemostasis valve is connected to the retrograde arm of the Y-connector by a first hemostasis valve connector, wherein the second hemostasis valve is connected to a proximal end of an extension line by a second hemostasis valve connector and wherein a distal end of the extension line is connected to the antegrade arm of the Y-connector.

According to one aspect of the present invention, there is provided an apparatus as described above, wherein the first hemostasis valve is connected to the retrograde arm of the Y-connector by a first hemostasis valve connector and wherein the first hemostasis valve connector comprises a flashback tube.

According to another aspect of the present invention, there is provided an apparatus as described above, wherein the second hemostasis valve is connected to the retrograde arm of the Y-connector by a second hemostasis valve connector and wherein the second hemostasis valve connector comprises a flashback tube.

According to yet another aspect of the present invention, there is provided an apparatus as described above, further comprising a plug disposed within the antegrade lumen between the side port and the distal tip.

According to another aspect of the present invention, there is provided an apparatus as described above, wherein the retrograde guidewire comprises a curved distal hook section.

According to one aspect of the present invention, there is provided an apparatus as described above, wherein the antegrade guidewire comprises a curved distal hook section.

According to another aspect of the present invention, there is provided an apparatus as described above, wherein the deflection microcatheter comprises a curved distal section.

According to yet another aspect of the present invention, there is provided an apparatus as described above, further comprising a deflection catheter guide attached to the second hemostasis valve.

According to still yet another aspect of the present invention, there is provided an apparatus as described above, wherein a proximal end of the deflection microcatheter includes a winged grip adapted to engage a portion of the deflection catheter guide, whereby a rotational orientation of the deflection microcatheter is controllably selected by engaging the winged grip with the portion of the deflection catheter guide.

According to yet another aspect of the present invention, there is provided an apparatus as described above, further comprising a removable clip adapted to engage the deflection catheter guide, whereby the clip prevents distal advancement of the deflection microcatheter when the clip is engaged with the deflection catheter guide.

According to still another aspect of the present invention, there is provided an apparatus as described above, wherein the clip includes a height that coincides with the second preselected distance by which the deflection microcatheter is advanced to cause a distal end thereof to project from the side port.

According to yet still another aspect of the present invention, there is provided an apparatus as described above, wherein a distal end of the deflection microcatheter is adapted to project from the side port in a lateral direction.

### BRIEF DESCRIPTION OF THE FIGURES

The accompanying drawings, which are incorporated in and constitute a part of the specification, illustrate various example apparatuses, systems, methods, and so on, that illustrate various example embodiments of aspects of the invention. It will be appreciated that the illustrated element boundaries (e.g., boxes, groups of boxes, or other shapes) shown in the Figures represent one example of the boundaries. One of ordinary skill in the art will appreciate that one element may be designed as multiple elements or that multiple elements may be designed as one element. An element shown as an internal component of another element may be implemented as an external component and vice versa.

Exemplary embodiments of the present invention shown in the Figures may not necessarily be drawn to scale, with emphasis instead being placed on illustrating the various aspects and features of the illustrated embodiments. In addition, one or more hidden elements may be removed from certain Figures in order to enhance the clarity and understanding of the remaining elements of the various embodiments of the present invention shown in the Figures. Common reference numerals are used among the Figures to depict features or elements that are common to the various embodiments of the present invention.

FIG. 1 is a perspective view of an apparatus according to one embodiment of the present invention, showing the apparatus in an assembled configuration;

FIG. 2 is an overhead plan view of the apparatus of FIG. 1, in which the various components of the apparatus are shown to all lie, generally, in the same plane;

FIG. 3 is an exploded overhead plan view of the apparatus of FIG. 1, showing the apparatus in a disassembled configuration;

FIG. 4A is a perspective view of a retrograde dilator component of the apparatus of FIG. 1;

FIG. 4B is a front view of the retrograde dilator of FIG. 4A;

FIG. 4C is a front cross-sectional view of a distal portion of the retrograde dilator of FIG. 4A, taken along a vertical section plane lying along a central longitudinal axis of the retrograde dilator;

FIG. 4D is a cross-sectional view of the retrograde dilator of FIG. 4C, taken along a vertical section plane 4D-4D lying perpendicular to the central longitudinal axis of the retrograde dilator;

FIG. 4E is a cross-sectional view of the retrograde dilator of FIG. 4C, taken along a vertical section plane 4E-4E lying perpendicular to the central longitudinal axis of the retrograde dilator;

FIG. 4F is a close-up cross sectional view of the distal portion of the retrograde dilator of FIG. 4C taken along a vertical section plane lying along a central longitudinal axis of the retrograde dilator;

FIG. 5A is a partially-exploded perspective view of the retrograde dilator of FIG. 4A;

FIG. 5B is a perspective view of a Y-connector component of the retrograde dilator of FIG. 5A;

FIG. 5C is a front view of the Y-connector component of FIG. 5B, in which certain hidden features thereof have been shown in dashed lines;

FIG. 5D is a perspective view of the Y-connector component of FIG. 5B;

FIG. 5E is a perspective view of the Y-connector component of FIG. 5B;

FIG. 5F is a perspective view of the Y-connector component of FIG. 5B;

FIG. 6A is a perspective view of first and second hemostasis valve connector components of the apparatus of FIG. 1;

FIG. 6B is a partially-exploded view of the hemostasis valve connector components of FIG. 6A;

FIG. 7A is a perspective view of an extension line component of the apparatus of FIG. 1;

FIG. 7B is a partially-exploded view of the extension line component of FIG. 7A;

FIG. 8 is a partially-exploded perspective view of the retrograde dilator, the first and second hemostasis valve connector and the extension line components of the apparatus of FIG. 1;

FIG. 9A is an overhead plan view of a retrograde guidewire component of the apparatus of FIG. 1;

FIG. 9B is an overhead plan view of an antegrade guidewire component of the apparatus of FIG. 1;

FIG. 10A is an overhead plan view of an antegrade deflection catheter component of the apparatus of FIG. 1;

FIG. 10B is a close-up overhead plan view of a proximal end of the antegrade deflection catheter of FIG. 10A, in which certain hidden features thereof have been shown in dashed lines;

FIG. 10C is a cross-sectional view of the antegrade deflection catheter of FIG. 10B, taken along a vertical section plane 10C-10C lying perpendicular to a central longitudinal axis of the antegrade deflection catheter;

FIG. 10D is a cross-sectional view of the antegrade deflection catheter of FIG. 10B, taken along a vertical section plane 10D-10D lying perpendicular to a central longitudinal axis of the antegrade deflection catheter;

FIG. 10E is a perspective view of a winged grip component of the antegrade deflection catheter of FIG. 10A, in which certain hidden features thereof have been shown in dashed lines;

FIG. 11A is a perspective view of the antegrade deflection catheter of FIG. 10A, into which the antegrade guidewire of FIG. 9B has been pre-loaded;

FIG. 11B is a cross-sectional view of the antegrade deflection catheter of FIG. 11A, taken along a vertical section plane 11B-11B lying perpendicular to a central longitudinal axis of the antegrade deflection catheter;

FIG. 11C is a close-up perspective view of a proximal end of the antegrade deflection catheter of FIG. 11A;

FIG. 11D is a close-up perspective view of a distal end of the antegrade deflection catheter of FIG. 11A;

FIG. 12A is a perspective view of a deflection catheter guide of the apparatus of FIG. 1;

FIG. 12B is an exploded perspective view of the deflection catheter guide of FIG. 12A;

FIG. 13A is a side elevation view of a guide body component of the catheter guide of FIG. 12A;

FIG. 13B is a rear perspective view of the guide body of FIG. 13A;

FIG. 13C is a front perspective view of the guide body of FIG. 13A;

FIG. 14A is a rear perspective view of certain components of the apparatus of FIG. 1, in which the guide body of FIG. 13A is shown in spaced relation to the hemostasis valve component of the second hemostasis valve connector of FIG. 6A;

FIG. 14B is a rear perspective view of certain components of the apparatus of FIG. 1, in which the guide body of FIG. 13A is shown attached to the hemostasis valve component of the second hemostasis valve connector of FIG. 6A;

FIG. 15A is a front perspective view of the guide body of FIG. 13A;

FIG. 15B is a bottom perspective view of the guide body of FIG. 13A;

FIG. 15C is a rear elevation view of the guide body of FIG. 13A;

FIG. 16A is a front perspective view of certain components of the apparatus of FIG. 1, in which the guide body of FIG. 13A is shown attached to the hemostasis valve component of the second hemostasis valve connector of FIG. 6A, and in which the antegrade deflection catheter of FIG. 11A (with the antegrade guidewire shown pre-loaded therein) is shown partially loaded into the apparatus of FIG. 1;

FIG. 16B is a close-up front perspective view of certain components of the apparatus of FIG. 1, in which the guide body of FIG. 13A is shown attached to the hemostasis valve component of the second hemostasis valve connector of FIG. 6A, and in which the antegrade deflection catheter of FIG. 11A (with the antegrade guidewire shown pre-loaded therein) is shown fully loaded into the apparatus of FIG. 1;

FIG. 16C is a close-up from perspective view of a distal end of the antegrade deflection catheter of FIG. 11A (with the antegrade guidewire shown pre-loaded therein) protruding from a side port of the retrograde dilator of FIG. 4A;

FIG. 17A is a front perspective view of a clip component of the deflection catheter guide of FIG. 12A;

FIG. 17B is a bottom perspective view of the clip of FIG. 17A;

FIG. 17C is an overhead plan view of the clip of FIG. 17A;

FIG. 18A is a partially-exploded front perspective view of certain components of the apparatus of FIG. 1, in which the guide body of FIG. 13Aa is shown in attached to the hemostasis valve component of the second hemostasis valve connector of FIG. 6A, and in which the clip of FIG. 17A is shown in spaced relation to the hemostasis valve component of the second hemostasis valve connector of FIG. 6A;

FIG. 18B is a front perspective view of certain components of the apparatus of FIG. 1, in which the guide body of FIG. 13A is shown attached to the hemostasis valve component of the second hemostasis valve connect of FIG. 6A, and in which the clip of FIG. 17A is shown positioned within a channel of the guide body formed by opposing arms;

FIG. 18C is a front perspective view of certain components of the apparatus of FIG. 1, in which the guide body of FIG. 13A is shown attached to the hemostasis valve component of the second hemostasis valve connect of FIG. 6A, the clip of FIG. 17A is shown positioned within a channel of the guide body formed by opposing arms and the antegrade advancement sub-assembly of FIG. 11A is shown in spaced relation to the hemostasis valve component of the second hemostasis valve connect of FIG. 6A; and,

FIG. 18D is a front perspective view of certain components of the apparatus of FIG. 1, in which the guide body of FIG. 13A is shown attached to the hemostasis valve component of the second hemostasis valve connect of FIG. 6A, the clip of FIG. 17A is shown positioned within a channel of the guide body formed by opposing arms and the antegrade advancement sub-assembly of FIG. 11A is shown in a pre-procedure retracted position relative to the hemostasis valve component of the second hemostasis valve connect of FIG. 6A.

### DETAILED DESCRIPTION OF THE PREFERRED EMBODIMENTS

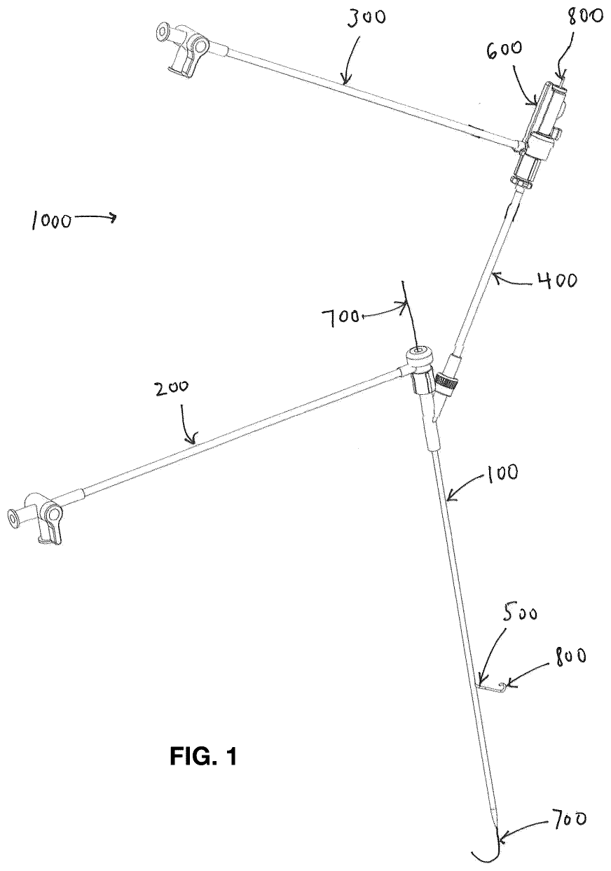

With reference to FIGS. 1-3, an apparatus 1000 according to one embodiment of the present invention is shown in an assembled, deployed configuration. A method of providing antegrade advancement of a medical device, such as a guidewire, catheter, or the like, through a patient's vasculature into which retrograde access to an artery thereof has been made and through which the medical device is introduced, by an apparatus, such as, for example, the apparatus 1000 shown in FIGS. 1-3, as well as methods for using an apparatus, such as the apparatus 1000 shown in FIGS. 1-3, to achieve antegrade advancement of a medical device while utilizing retrograde access to an artery of the patient's vasculature, are described in greater detail below.

The apparatus 1000 includes a retrograde dilator 100, first and second hemostasis valve connectors 200, 300, respectively, extension line 400, antegrade deflection microcatheter 500, deflection catheter guide 600, retrograde guidewire 700 and antegrade guidewire 800. Each of these components will be described, along with the referenced figures, in greater detail below in the following detailed description of the preferred embodiments.

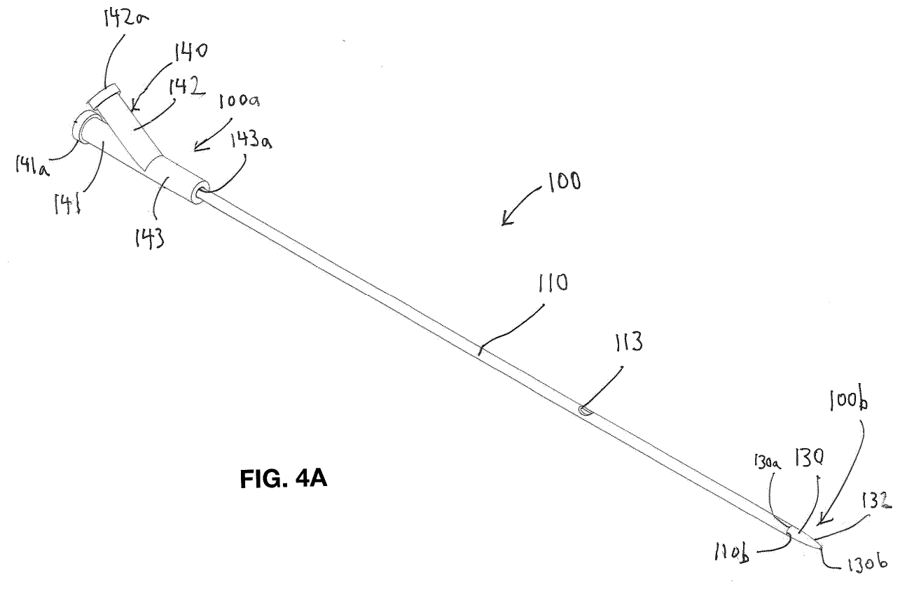

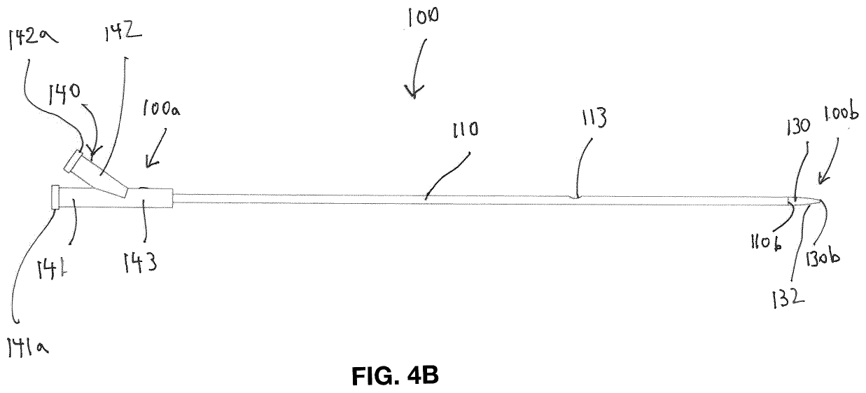

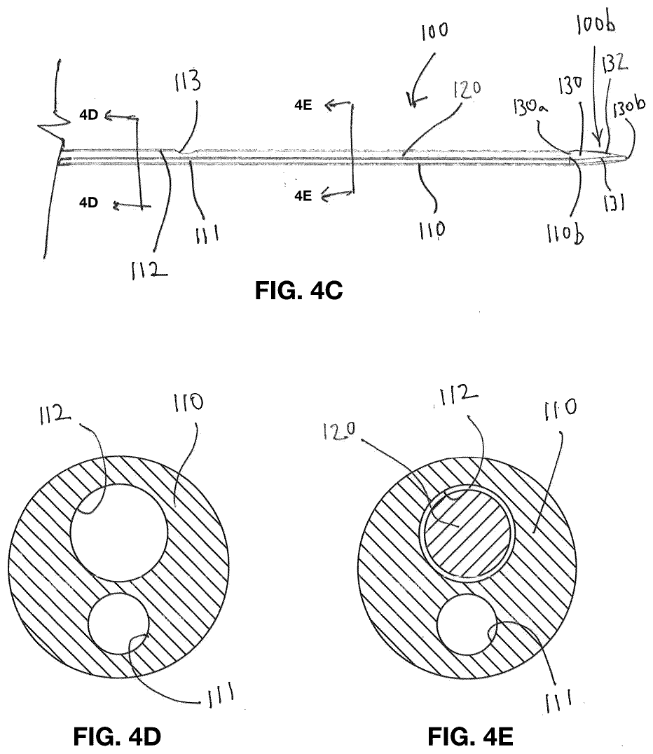

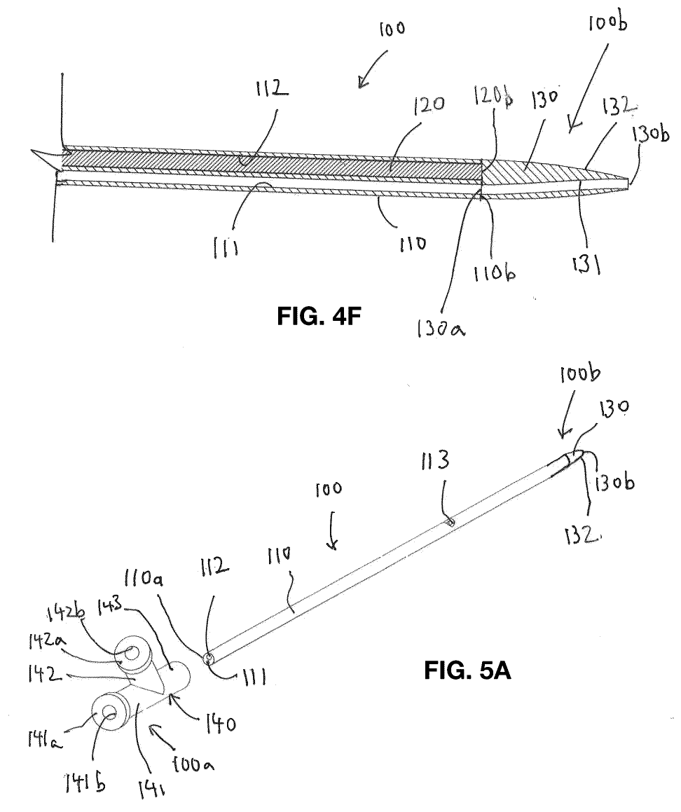

With additional reference now to FIGS. 4A-4C, the retrograde dilator 100 of the apparatus 1000 is shown having a proximal portion 100a and a distal portion 100b opposite the proximal portion 100a. The retrograde dilator 100 comprises a dual lumen main body 110 extending, generally, from the proximal portion 100a to the distal portion 100b of the dilator 100, wherein the dual lumen main body 110 comprises a retrograde lumen 111 and an antegrade lumen 112, both extending continuously therethrough along a longitudinal axis of the main body 110. Main body 110 includes a side port 113 positioned approximately one-third of the length of the main body 110 proximal of the distal end 100b of the dilator 100 and communicating with antegrade lumen 112. Dilator 100 includes antegrade lumen plug 120 positioned within the antegrade lumen 112 and extending between the side port 113 and a distal end 110b of the main body 110.

Main body 110 is constructed, preferably, from a polymer, such as, for example, high density polyethylene (“HDPE”), that has sufficient compliance/elasticity so as to allow the main body 110 to bend and return to its original shape without kinking, yet with sufficient column strength to provide sufficient “pushability” to function as described herein. Low density polyethylene (“LDPE”) may be used for thin-wall structures, but may need to be reinforced to prevent kinking and to provide sufficient column strength. In addition, the preferred material, such as HDPE, preferably has a sufficiently-low coefficient of friction to permit the main body 110 to be introduced into a patient's body and vasculature, possibly through an introducer sheath, without causing unnecessary or undesirable resistance from the patient's body tissue, etc. The main body 110 is formed as a coextrusion with retrograde lumen 110 and antegrade lumen 112 formed therein according to conventional extrusion techniques. Main body 110 is formed and then cut to length (a so-called “blunt-cut co-extrusion”) to be, according to one embodiment of the present invention, 100-150 cm in length.

With additional reference now also to FIG. 4F, antegrade lumen plug 120 is formed as a simple monofilament extrusion of HDPE and cut to length to be, according to one embodiment of the present invention, 30-40 cm in length. Antegrade lumen plug 120 is then inserted into the distal end 110b of the main body 110 until its own distal end 120b is positioned roughly near the distal tip 130b of the distal end 130 such that it does not extend distally therefrom.

Once positioned properly within the antegrade lumen 112, the distal end 110b of the main body 110 is “tipped” according to conventional techniques whereby the distal end 110b is placed within a mold having a tapered shape conforming to the desired shape of the forwardly-tapered front end 132 of the distal tip 130. The mold is then heated and compressed with mating parts such that the distal tip 130, as shown, results in a tapered shape in which the tip lumen 131 forms a continuous path from the retrograde lumen 111 of the main body 110 therethrough and the antegrade lumen portion thereof (not shown) is effectively closed by the melting of a distal segment of the plug 120, forming an anchor and back-stop against which distal movement of the plug 120 within the antegrade lumen 112 of the main body 1100 is prevented.

Next, the side port 113 is formed in the main body 110 located roughly 30-40 cm proximal to the distal tip 130b by applying a conventional heated forming tool (not shown) against the sidewall of the main body 110 adjacent the radial location of the antegrade lumen 112 and at the longitudinal location of a proximal end of the plug 120 dwelling within the antegrade lumen 112. Under the influence of heat and pressure, the forming tool is pressed against the sidewall of the main body 110 until it pierces the sidewall, thereby gaining access to the antegrade lumen 112. The forming tool is shaped, oriented and advanced against the main body 110 at angles relative thereto so that a ramp feature (not shown) may be formed in the proximal end of the plug 120, which serves the purpose of assisting advancement of the antegrade guidewire during use.

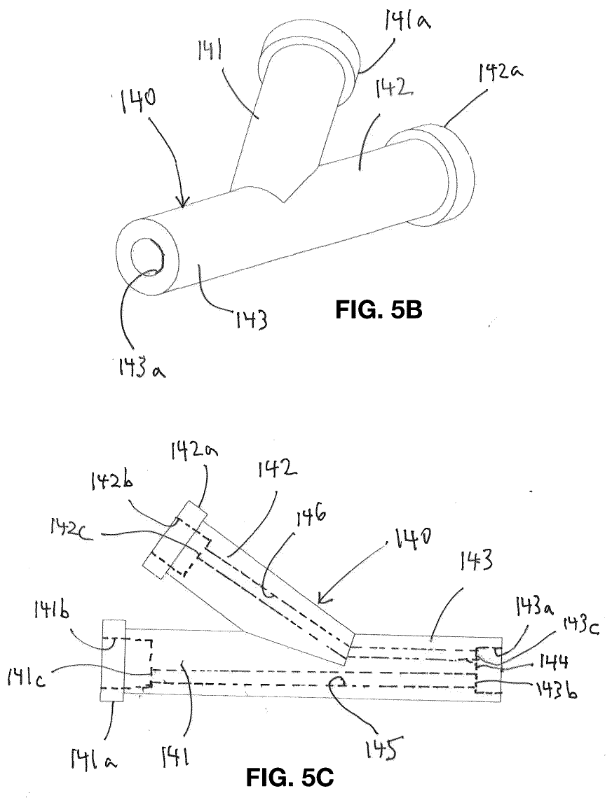

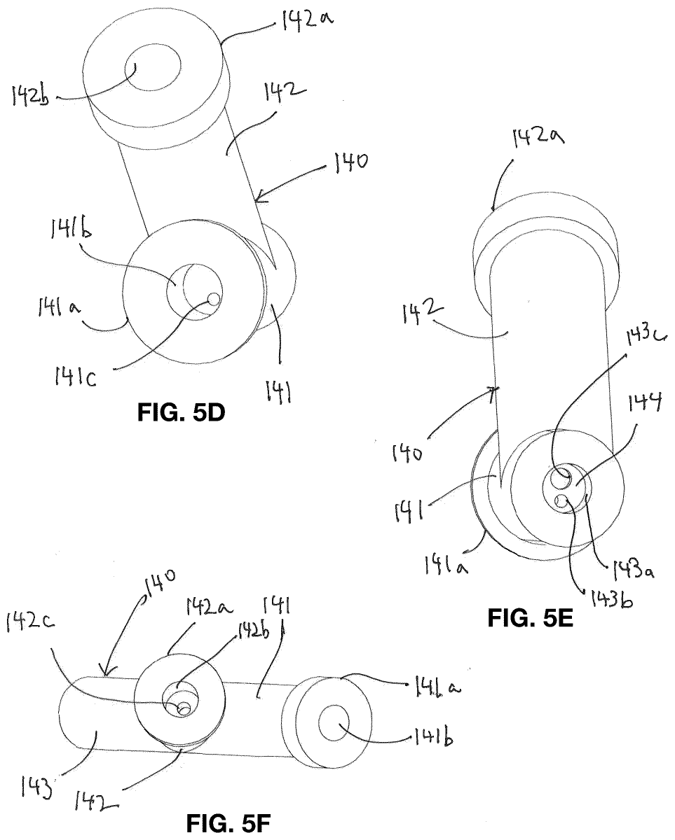

From these drawings and this description, it will be understood that retrograde lumen 111 and antegrade lumen 112 both, as formed, extend continuously along the full length of the main body 110 from its proximal end 110a (FIG. 5A) to its distal end 110b. As such, retrograde lumen 111 is open at both the proximal end 110a (FIG. 5A) and the distal end 110c of the main body 110, whereas plug 120 obstructs the antegrade lumen 112 distal of the side port 113. And while FIG. 4E suggests that an annular space is permitted between an inner surface of antegrade lumen 112 and an outer surface of plug 120, this is for illustration only. It is preferred that there is an interference fit between the outer surface of the plug 120 and the inner surface of the antegrade lumen 112 such that the plug 120 completely obstructs antegrade lumen 112, and is immovable therein, distal the side port 113 through the rest of its distal length. As such, due to plug 120 being positioned within the antegrade lumen 112 between the side port 113 and distal end 110b of the main body 110, antegrade lumen 112 is obstructed by plug 120 at its distal end.

Retrograde dilator 100 also includes, at the distal end 100b thereof, a distal tip 130 with a tip lumen 131 passing therethrough from an open proximal 130a to an open distal end 130b thereof. Preferably, as shown, distal tip 130 includes a forwardly-tapered front end 132. Distal tip 130 may be integrally-formed with the main body 110 or may be formed in a separate process, shaped and then affixed to the distal end 110b of the main body 110 using conventional techniques such as heat bonding, welding, adhesive affixation, etc. While distal tip 130 has been described as having a forwardly-tapered front-end 132, it may alternatively have a blunt front tip.

With reference now also to FIG. 4F, distal tip lumen 131 is aligned with retrograde lumen 111 at the proximal end 130a of the distal tip 130 and is centrally-located (i.e., aligned with the longitudinal axis of the main body 110) at the distal end 130b thereof, thereby providing a smooth transition from the off-axis retrograde lumen 111 to an on-axis distal exit port formed by the open distal tip lumen 131 at the distal end 130b of the distal tip 130. The flat, proximal end 130a of the distal tip 130 abuts the plug 120 at the distal end 120b thereof, thereby further preventing plug 120 from moving distally within the antegrade lumen 112.

Referring back to FIGS. 4A and 4B, as well as to FIGS. 5A-5F, retrograde dilator 100 includes a dual-lumen Y-connector 140 defining the proximal end 100a of the retrograde dilator 100, in which the Y-connector 140 comprises an retrograde arm 141, an antegrade arm 142 and an outlet arm 143. Y-connector 140 may be integrally formed by any conventional molding process. Retrograde arm 141 and antegrade arm 142 each include a proximal connector portion 141a, 142a, respectively, that comprises, preferably, a luer lock fitting onto which compatible connectors may be attached, as known to those of ordinary skill in the art, and for the purposes described hereinbelow.

Outlet arm 143 includes an outlet recess 143a into which the proximal end 110a of the main body 110 is inserted, and which may be affixed thereto, such as, for example, by heat bonding, welding or adhesive fixation. With reference specifically to FIG. 5E, outlet recess includes a distal-facing abutment 144, against which the proximal end 110a of the main body 110 abuts when it is inserted into the recess 143a. Similarly, retrograde arm 141 includes a recess 141b and antegrade arm 142 includes a recess 142b, wherein both recesses 141b, 142b are sized to receive compatible connectors as shown and described below. An opening 141c is provided in the recess 141b of the retrograde arm 141 and a first opening 143b is provided in the recess 143a of the outlet arm 143, between which a retrograde channel 145 is formed continuously through the body of the Y-connector 140. Similarly, an opening 142c is provided in the recess 142b of the antegrade arm 142 and a second opening 143c is provided in the recess 143a of the outlet arm 143, between which an antegrade channel 146 (separate from the retrograde channel 145) is formed continuously through the body of the Y-connector 140.

First opening 143b is formed, preferably, in, and positioned on, the face of the abutment 144 such that when the proximal end 110a of the main body 110 is inserted into, and affixed to, the recess 143a of the outlet arm 143, the first opening 143b is aligned with, and provides a continuous transition to, the retrograde lumen 111 of the main body 110. Similarly, second opening 143c is formed, preferably, in, and positioned on, the face of the abutment 144 such that when the proximal end 110a of the main body 110 is inserted into, and affixed to, the recess 143a of the outlet arm 143, the second opening 143c is aligned with, and provides a continuous transition to, the antegrade lumen 112 of the main body 110.

The dual lumen dilator 100 configured as described above, then, allows for certain medical devices, such as, for example, the retrograde guidewire 700 described below, to be inserted into the opening 141c of the recess 141b of the retrograde arm 141 of the Y-connector 140 and advanced through the retrograde channel 145 of the Y-connector 140, through the retrograde lumen 111 of the main body 110, through the distal tip lumen 131 and out through the open distal end 130b of the distal tip 130. At the same time, and separately therefrom, the dual lumen dilator 100 configured as described above, allows for certain other medical devices, such as, for example, the antegrade guidewire 800 described below, to be inserted into the opening 142c in the recess 142b of the antegrade arm 142 of the Y-connector 140 and advanced through the antegrade channel 146 of the Y-connector 140, through the antegrade lumen 112 of the main body 110 and out through the open side port 113 of the main body.

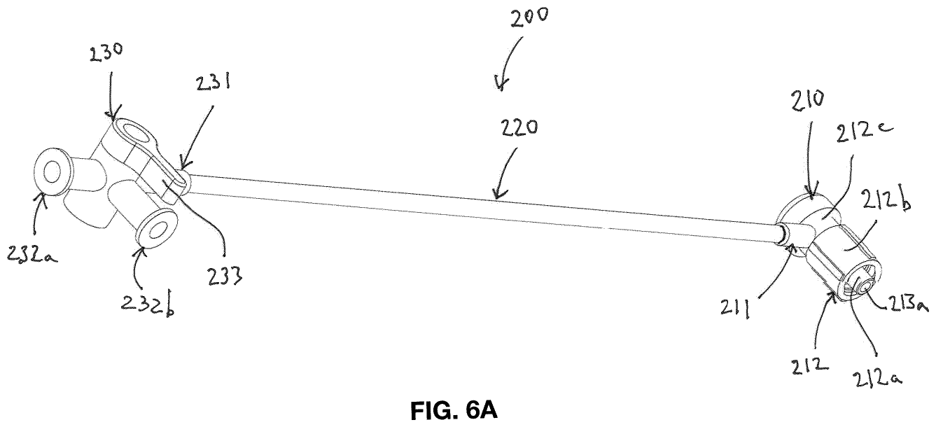

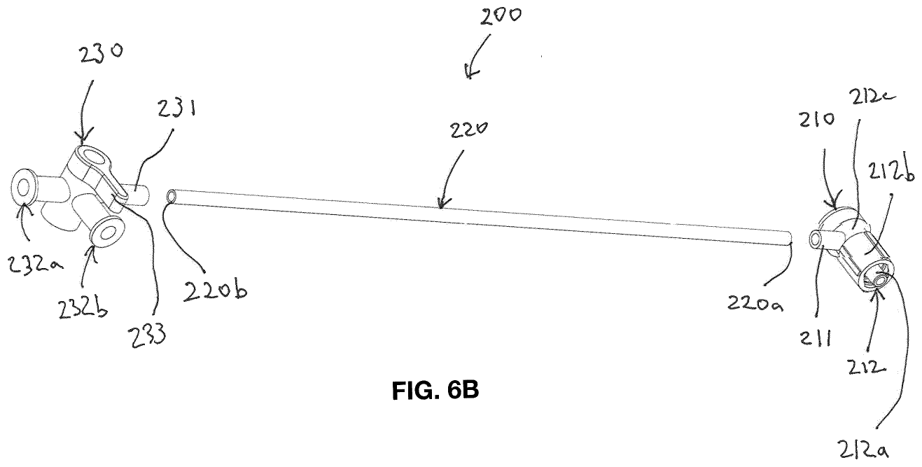

With reference now to FIGS. 6A and 6B, the first hemostasis valve connector 200 according to one embodiment of the present invention is shown comprising a conventional hemostasis valve 210 with side port 211, a flexible, preferably at least partially transparent flashback tube 220 connected at one end 220a thereof to the side port 211 of the hemostasis valve 210, and a conventional valve 230 having a side port 231 connected to another end 220b of the flashback tube 220. Hemostasis valve 210 includes a connector 212 configured (i.e., preferably compatible as a twist-type luer lock connector) to sealingly engage the proximal connector portion 141a of the retrograde arm 141 of the retrograde dilator Y-connector 140. More specifically, connector 212 includes a stem 212a sized to be received by the recess 141b of the retrograde arm 141 around which a lock 212b is rotatably connected. Lock 212b includes an inner threaded surface configured to threadingly receive proximal connector portion 141a. As lock is twisted around its access (which it shares with the access of the stem 212a), threads draw the proximal connector portion 141a along the axis toward the main body 212c of the hemostasis valve 210. With reference also back to FIG. 1, hemostasis valve 210 includes a conventional inlet 213 that is sized to receive, for example, the retrograde guidewire 700 according to one embodiment of the present invention, and includes a lumen 213 that passes through stem 212a. Inlet 213 is configured to maintain a seal around retrograde guidewire 700 as the guidewire 700 is advanced therethrough for purposes described in greater detail below.

Valve 230 is shown having first and second connector ports 232a, 232b, each having a conventional luer lock fitting adapted to be connectable to other medical devices and supplies, such as tubing, etc., connectable to, for example, an IV bag containing, for instance, sterile saline. Valve 230 also includes multi-position stopcock 233 which, as shown, may be adapted to be moved to one of three positions: a first open position in which the first connector port 232a (and anything that might be connected to first connector port 232a) is in fluid communication, through the main body of the valve 230, with side port 231; a second open position in which the second connector port 232b (and anything that might be connected to second connector port 232b) is in fluid communication, through the main body of the valve 230, with side port 231; and, a third closed position (shown) in which side port 231 is not in fluid communication with cither first or second connector ports 232a, 232b, respectively. While valve 230 is shown with first and second connector ports 232a, 232b, respectively, it may alternatively include only one port or any number of ports. With reference now back to FIGS. 1-3, second hemostasis valve connector 300 is, in one embodiment of the present invention, identical to first hemostasis valve connector 200, and as such, like parts are numbered consistently throughout the figures with like reference numbers.

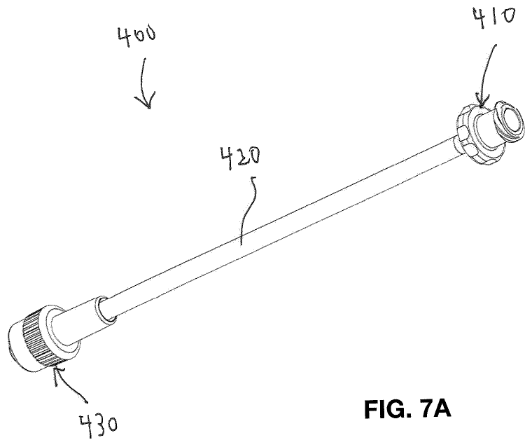

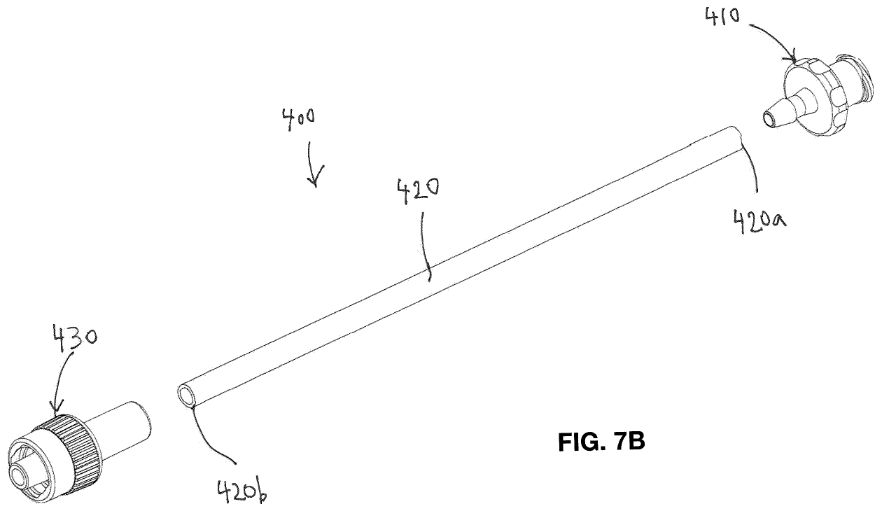

With reference to FIGS. 7A and 7B, the extension line 400 according to one embodiment of the present invention is shown comprising a female luer connector 410, a flexible, preferably at least partially transparent extension tube 420 connected at one end 420a thereof to the female luer connector 410, and a male luer connector 430 connected to another end 420b of the extension tube 420.

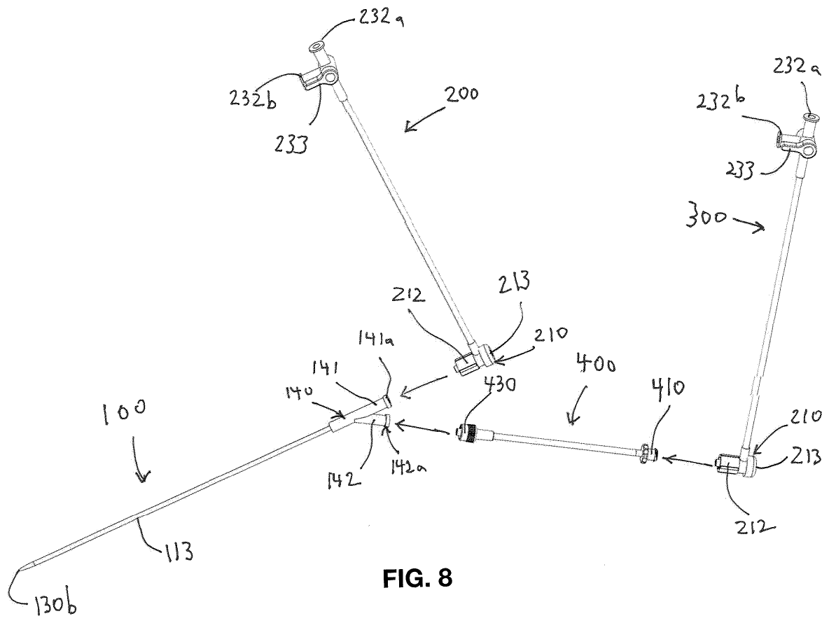

Referring now to FIG. 8, the apparatus 1000 is partially assembled by first connecting the connector 212 of the hemostasis valve 210 of the first hemostasis valve connector 200 to the proximal connector portion 141a of the retrograde arm 141 of the retrograde dilator Y-connector 140. Next, the male luer connector 430 of the extension line 400 is connected to the proximal connector portion 142a of the antegrade arm 142 of the retrograde dilator Y-connector 140. Finally, the connector 212 of the hemostasis valve 210 of the second hemostasis valve connector 300 is connected to the female luer connector 410 of the extension line 400. According to some aspects of the present invention, extension line 400 may be omitted altogether, in which case, the connector of the hemostasis valve 210 of the second hemostasis valve connector 300 may be connected directly to the connector portion 142a of the antegrade arm 142 of the retrograde dilator Y-connector 142.

A continuous, sealed “retrograde” fluid path is thus selectively formed between the first and second connector ports 232a, 232b, respectively, of the first hemostasis valve connector 200 and the open distal end 130b at the distal tip 130 of the retrograde dilator 100, specifically, via the retrograde lumen 111 (FIG. 4F) that passes though the main body 110 of the retrograde dilator 100. Inlet 213 of the hemostasis valve 210 of the first hemostasis valve connector 200 joins the continuous, sealed “retrograde” fluid path at the connector 212 of the first hemostasis valve connector 200, and as such, the inlet 213 also is open to the open distal end 130b at the distal tip 130 of the retrograde dilator 100, via the retrograde lumen 111 (FIG. 4F). As such, a guidewire, such as the retrograde guidewire 700 according to one embodiment of the present invention, or other medical device, such as, for example, a catheter, can be inserted into the inlet 213 of the hemostasis valve 210 of the first hemostasis valve connector 200 and advanced through the retrograde dilator 100 until, if long enough, is exits distally out the open distal end 130b at the distal tip 130 of the retrograde dilator 100.

Similarly, a continuous, sealed “antegrade” fluid path is selectively formed between the first and second connector ports 232a, 232b, respectively, of the second hemostasis valve connector 300 and the side port 113 of the retrograde dilator 100, specifically, via the antegrade lumen 112 (FIG. 4F) that passes though the main body 110 of the retrograde dilator 100. Inlet 213 of the hemostasis valve 210 of the second hemostasis valve connector 300 joins the continuous, sealed “antegrade” fluid path at the connector 212 of the second hemostasis valve connector 300, and as such, the inlet 213 also is open to the side port 113 of the retrograde dilator 100, via the antegrade lumen 112 (FIG. 4F). As such, a guidewire, such as the antegrade guidewire 800 according to one embodiment of the present invention, or other medical device, such as, for example, a catheter, can be inserted into the inlet 213 of the hemostasis valve 210 of the second hemostasis valve connector 300 and advanced through the retrograde dilator 100 until, if long enough, is exits out the side port 113 of the retrograde dilator 100.

As will be described in greater detail below, the position, orientation and distal advancement of the antegrade guidewire 800 provides certain advantages and benefits to the method of using an apparatus, such as, for example, the apparatus 1000 according to one embodiment of the present invention to provide access to the vasculature of a patient wherein the apparatus 1000 is provided to facilitate access to the patient's vasculature in the antegrade direction from a generally-retrograde-oriented access point.

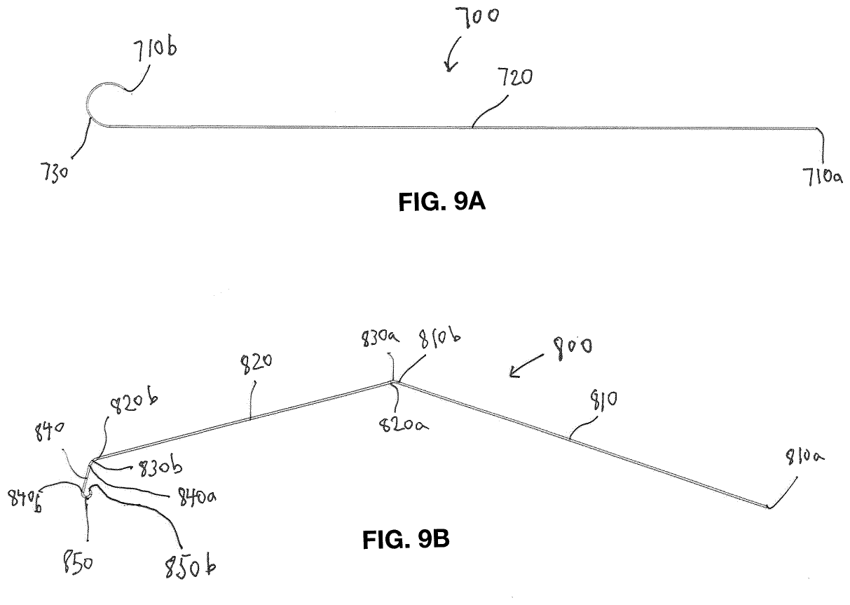

Referring to FIG. 9A, the retrograde guidewire 700 is a conventional fixed core guidewire formed from a shape memory material such as nitinol. Alternatively, guidewire 700 may be formed from conventional materials such as stainless steel. The guidewire 700 may be uncoated, although preferably it is coated with a lubricious agent to facilitate movement thereof as described herein. When relaxed, retrograde guidewire 700, generally, lies entirely in a plane and comprises a proximal tip 710a, a generally straight proximal portion 720 and a curved distal hook 730 terminating in a distal tip 710b. The radius of the distal hook 730, and extent to which is curves back on itself can be optimized for the purposes described herein, but preferably is configured as follows.

With additional reference to FIGS. 1 and 4A-5F, as well as the preceding description, those of ordinary skill in the art will understand that retrograde guidewire 700 can be straightened out, for example, to permit its insertion into, and advancement through, the retrograde dilator 100, via the first hemostasis valve 210, through the retrograde lumen 111 and out the open distal end 130b of the distal tip 130, after which, if left unconstrained, it will project distally of the distal tip 130 and return, at least partially, to its curved shape. Retrograde guidewire 700 has a length, preferably 12 inches, such that when the curved distal hook 730 projects distally from the distal tip 130 a distance sufficient for it to revert to its curved shape, a sufficient length of the straight proximal portion 720 projects proximally from the inlet 213 of the first hemostasis valve 210 such that it may be gripped by a user and either advanced further distally (thereby increasing the length of the curved distal hook 730 extending distally from the open distal end 130b of the distal tip 130), withdrawn proximally (thereby decreasing the length of the curved distal hook 730 extending distally from the open distal end 130b of the distal tip 130) or rotated, thereby altering the orientation of the plane in which the curved distal hook 730 lies relative to the place in which, for example, the retrograde dilator 100 lies. Because the retrograde guidewire 700 is constructed from a shape memory material and because the retrograde dilator 100 is constructed from the relatively stiff material, withdrawing the curved distal hook 730, or a portion thereof, proximally back into the retrograde dilator 100 will cause the portion of the curved distal hook 730 to be constrained within the retrograde lumen 111 to straighten out. Further advancement thereof distally will cause any portion of the curved distal hook 730 projecting distally from the open distal end 130b of the distal tip 130, thus unconstrained, to revert back to its curved shape and reoriented either axially or radially as described above.

With reference now to FIG. 9B, antegrade guidewire 800 has a similar construction to the retrograde guidewire 700 as it relates to the materials chosen therefor, as well as any coatings used therewith. When relaxed, antegrade guidewire 800 also lies in a plane and comprises a proximal straight section 810 having a proximal tip 810a and a distal end 810b connected to a proximal end 820a of a distal straight section 820 by a first curved section 830a curved in a first direction, and a distal extension 840 having a proximal end 840a connected to a distal end 820b by a second curved section 830b curved in the first direction, and a distal hook 850 extending from a distal end 840b of the extension 840 and curved in the first direction. As will be described in greater detail below, antegrade guidewire 800 can be straightened out, such as, for example, by inserting it into, and advancing it through, the antegrade lumen 112 of the retrograde dilator 100, and returned to the curved configuration when it is removed therefrom.

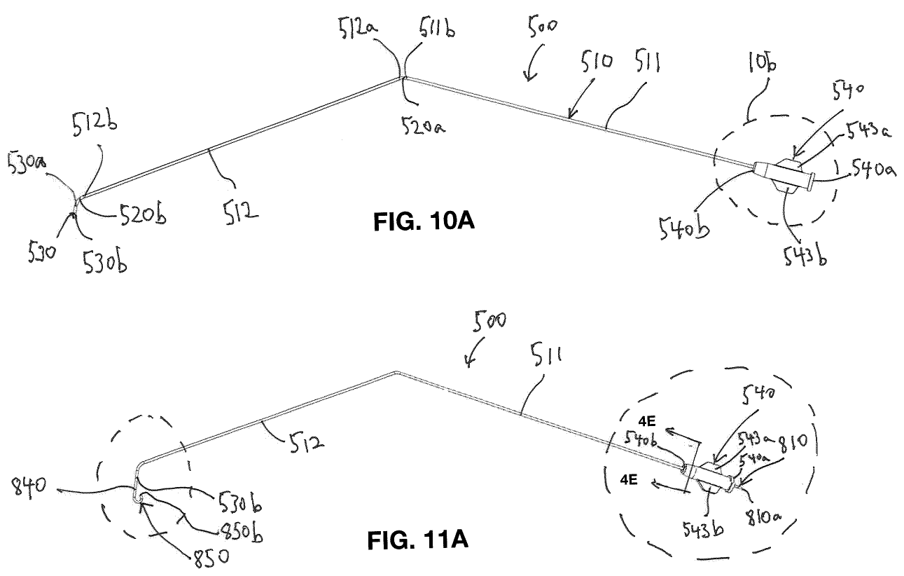

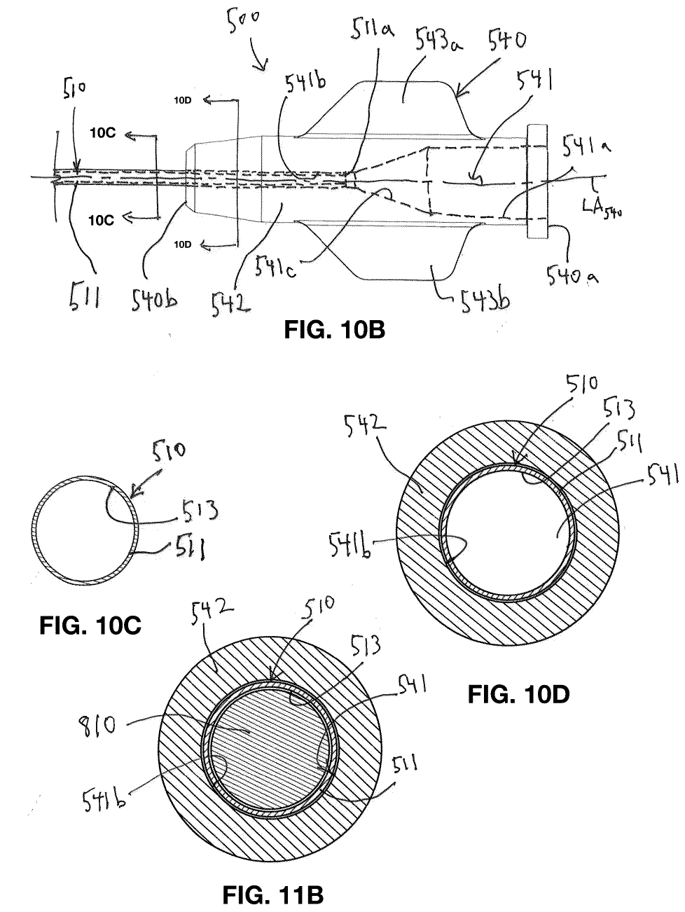

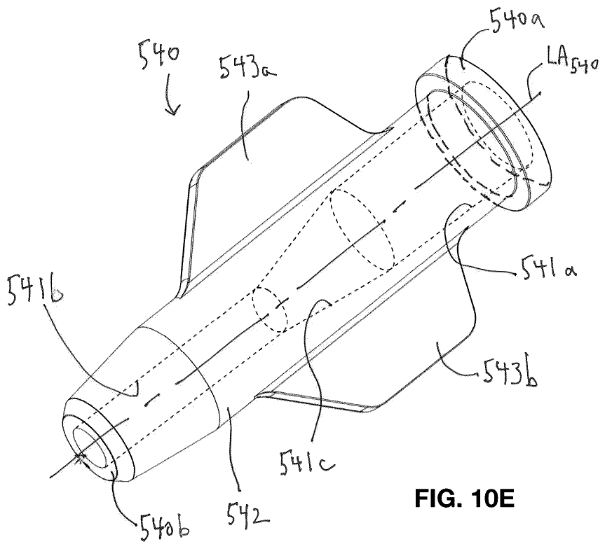

FIG. 10A depicts the antegrade deflection microcatheter 500 of the apparatus 1000 according to one embodiment of the present invention. Microcatheter 500 is constructed from a flexible material, such as PEEK, and includes a relaxed shape configuration that resembles the relaxed shape configuration of the antegrade guidewire 800 (FIG. 9B). When relaxed, microcatheter 500 lies entirely in a plane and includes a main body 510 comprising a proximal straight section 511 having a proximal tip 511a (FIG. 10B) and a distal end 511b connected to a proximal end 512a of a distal straight section 512 by a first curved section 520a curved in a first direction, and, alternatively, a distal extension 530 having a proximal end 530a connected to a distal end 512b by a second curved section 520b curved in the first direction and an open distal end 530b. If the microcatheter 500 is not provided with a distal extension 530, as shown, the distal end of the microcatheter 500 terminates with an open end of the second curved section 520b. Alternatively, microcatheter 500 may be formed as a two-piece construction in which the main body thereof is formed from a conventional polymer with a nitinol tip affixed to the distal end thereof.

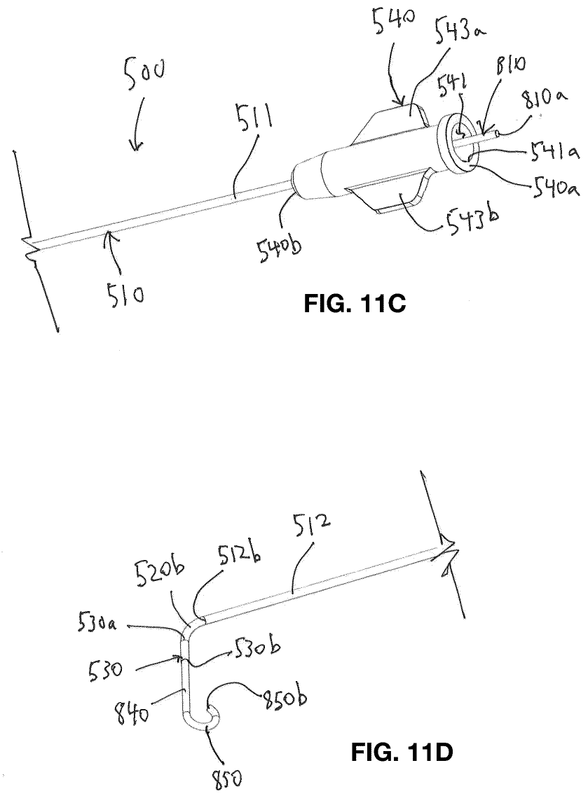

Referring now to FIGS. 10A, 10B, 10E and 11C, deflection microcatheter 500 also includes winged grip 540 having an open proximal end 540a, an open distal end 540b and a tapered lumen 541 extending therethrough. Lumen 541 includes a proximal portion 541a and a distal portion 541b communicating with the proximal portion 541a by tapered portion 541c therebetween. The distal portion 541b of the lumen 541 is sized to receive the proximal straight section 511 of the microcatheter main body 510 therein, forming a tight, preferably fixed, fit therebetween such that microcatheter main body 510 cannot be removed from the winged grip 540 once inserted and affixed thereto. Winged grip 540 includes a body portion 542 through which the lumen 541 extends and includes two, generally flat, wings 543a, 543b projecting therefrom. Preferably, wings 543a, 543b lie in a plane that intersects a longitudinal axis LA₅₄₀ of the winged grip 540 running longitudinally through the center axis of the body portion 542. Lumen portions 541a, 541b, 541c each extend along an axis that is coaxial with longitudinal axis LA₅₄₀. Preferably, two wings 543a, 543b, are provided, as shown, but alternatively, any number of wings can be provided and arranged around the body portion 542 of the winged grip.

Referring now specifically to FIG. 10a, and for the purposes described below, the plane in which the entire main body 510 of the microcatheter 500 lies, when in a relaxed condition, is also the plane in which wings 543a, 543b lie when the proximal straight section 511 of the microcatheter main body 510 is affixed to the distal portion 541b of the lumen 541. This provides a visual indication to the user as to the rotational orientation of the extension 530 by observing the rotational orientation of the wings 543a, 543b. That is, the user knows the orientation of the extension 530, even when the orientation of the extension 530 is not directly observable (e.g., after the extension 530 has been positioned within a patient's body), simply by observing the orientation of the wings 543a, 543b (i.e., the extension 530 and the wings 543a, 543b lie in the same plane).

FIG. 10C shows a cross-sectional view of the main body 510 of the microcatheter 500 at the proximal straight section 511 thereof taken along section line 10c-10c of FIG. 10B and shows that main body 511 includes a lumen 513 that extends continuously from the proximal tip 511a of the proximal straight section 511, distally therefrom through the first curved section 520a, through the distal straight section 512, through the second curved section 520b, through the extension 530 and is open at the distal open end 530b of the extension 530.

FIG. 10D shows a cross-sectional view of the body portion 542 of the winged grip 540 taken along section line 10d-10d of FIG. 10B and shows a slightly exaggerated arrangement in which the proximal straight section 511 of the microcatheter main body 510 has a diameter that, when inserted into the distal portion 541b of the lumen 541, an outer surface of the proximal straight section 511 is slightly spaced radially inwardly from the lumen 541. However, this is for illustration only. Preferably, there is an interference fit between the two such that the proximal straight section 511 of the microcatheter main body 510 cannot be easily removed from the distal portion 541b of the lumen 540. In addition, the two may, in fact, be bonded, such as, for example, by adhesive or by conventional thermal bonding.

Referring now to FIGS. 10B and 11C, the open proximal end 540a of the winged grip 540 exposes the proximal portion 541a of the lumen 541 proximally to the user and provides a large enough inlet into which the user can insert the distal tip 850b (FIG. 9B) of the distal hook 850 (FIG. 9B) (which has been manually straightened out against the bias of the shape memory material to urge the distal hook 850 to assume the shape shown in FIG. 9B) of the antegrade guidewire 800, into the open proximal end 541a of the lumen 541. Tapered portion 541c of the lumen 541 further guides advancement of the distal tip 850b smoothly into the lumen 513 of the microcatheter main body 510. From there, the user can advance the antegrade guidewire 800 through the entire main body 510 of the microcatheter 500 until the distal hook 850 (FIGS. 11A and 11C) is advanced distally out of the distal open end 530b of the extension 530 (FIG. 11A). As mentioned above, guidewire 800 may be coated (or alternatively, an inner surface of the lumen 513 may be coated) with a lubricious material so as to enhance advancement of the guidewire 800 within the microcatheter 500.

With additional reference now to FIG. 11A, after the guidewire 800 has been advanced into a “pre-loaded” position (such as shown in FIG. 11A), the proximal tip 810a of the guidewire 800 protrudes proximally from the open proximal portion 541a of the winged grip 540 by a distance sufficient to permit the user to grasp the proximal straight section 810 of the guidewire to permit further advancement distally, or withdrawal proximally, relative to the microcatheter 500. In addition, when in this “pre-loaded” position, the distal tip 850b of the guidewire 800 protrudes from the open distal end 530b of the microcatheter 500. At the same time, respective proximal straight sections 511, 810, first curved sections 520a, 830a, distal straight sections 512, 820, and second curved sections 520b, 830b, of the microcatheter 500 and the guidewire 800, respectively, are generally aligned such that the coincide with one another, generally, and lie in the same place when in a relaxed configuration. As thus shown and configured in FIGS. 1, 2 and 11A-11C, the antegrade deflection microcatheter 500 and the antegrade guidewire 800, together, are herein referred to as the “antegrade advancement sub-assembly”.

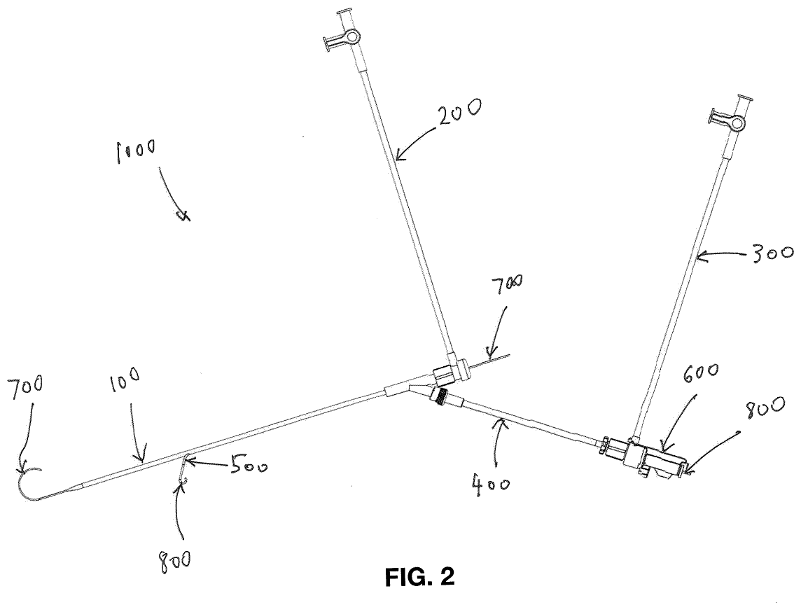

With specific, comparative, reference now to FIGS. 2 and 11A, one of ordinary skill in the art will appreciate that the configuration and orientation of the antegrade advancement sub-assembly when in the relaxed configuration (shown in FIG. 11A) is similar to the assembled configuration and orientation of the retrograde dilator 100, to which the extension line 400 has been attached as described above and shown in FIG. 2. Particularly, and for example, a distance between the first and second curved sections 830a, 830b, respectively, of the guidewire 800 (FIG. 9B) is generally the same as a distance between the first and second curved sections 520a, 520b, respectively, of the microcatheter 500 and is generally the same as a distance between a point at which the antegrade arm 142 of the Y-connector 140 meets the outlet arm 143 thereof and the side port 113 of the retrograde dilator 100. Similarly, an arc of curvature of the first curved section of the guidewire (FIG. 9B) is generally the same as an arc of curvature of the first curved section 520a of the microcatheter 500 and is generally the same as an angle formed between a central axis of the outlet arm 143 of the Y-connector 140 and a central axis of the antegrade arm 142 of the Y-connector 140.

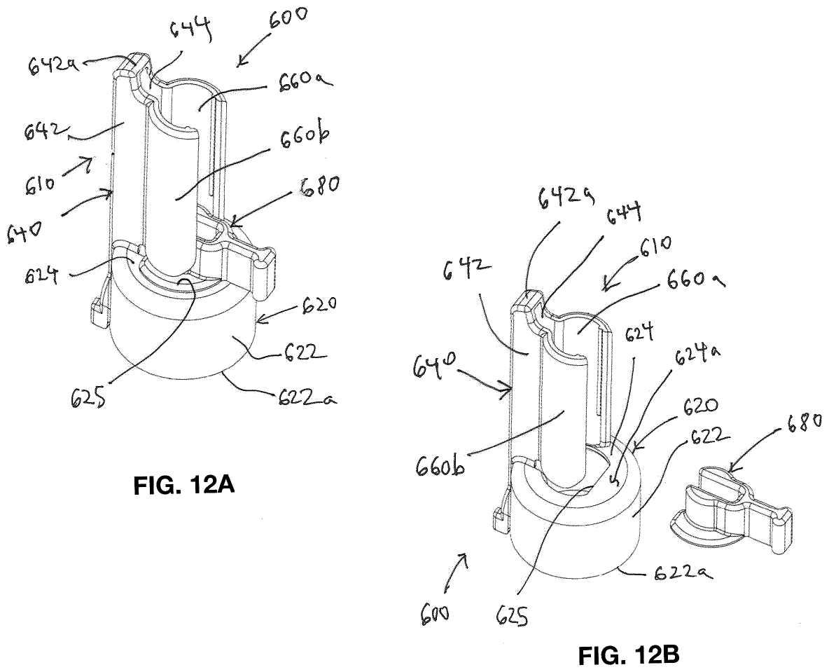

The deflection catheter guide 600 according to one embodiment of the present invention is shown in FIGS. 12A and 12B, in which the guide 600 is shown in an exploded view in FIG. 12B. Deflection catheter guide 600 comprises two main components: guide body 610 and advancement lock 680, which is removably positionable relative to guide body 610 as shown and described in more detail below.

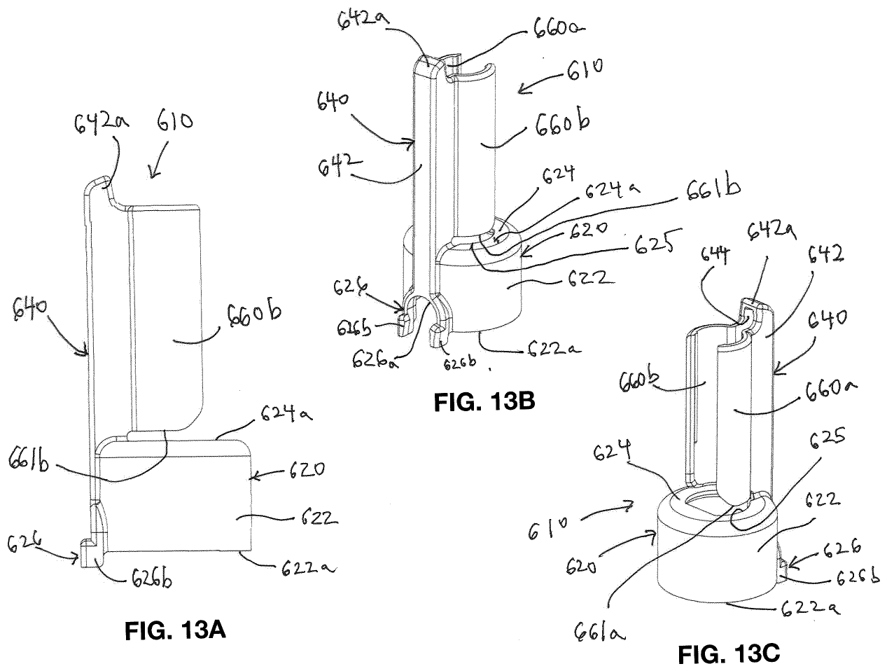

With additional reference to FIGS. 13A-13C, the guide body 610 includes a collar 620 comprising a cylindrical side wall 622 with an open bottom end 622a and top wall 624 opposite the open bottom end 622a, in which the top wall 624 forms a flat surface 624a lying in a plane that is perpendicular to a central axis of the side wall 622. Top wall 624 includes an opening 625 in the center thereof. The side wall 622 includes a snap connector 626 defined by a cutout 626a in the side wall 622 positioned at and projecting from the open bottom end 622a toward the top wall 624. Cutout 626a has a generally arcuate shape.

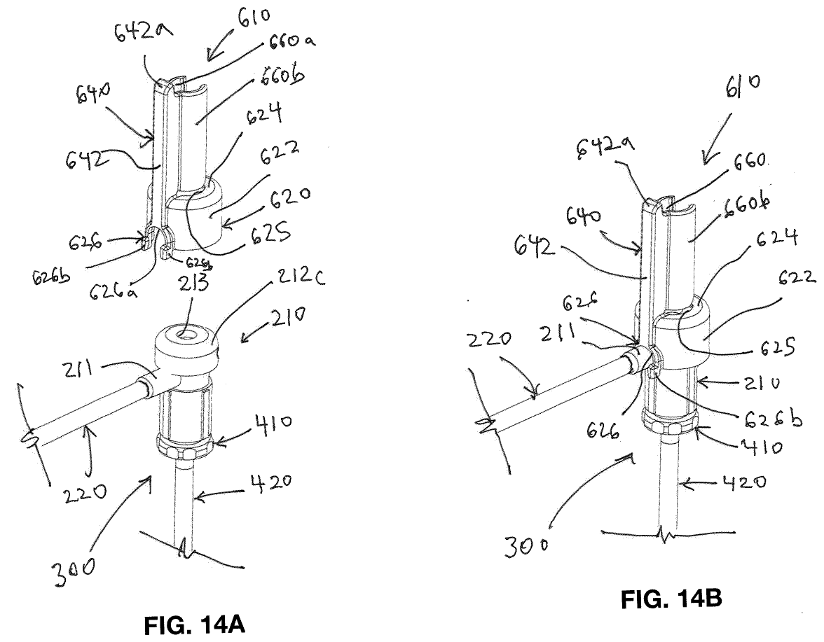

Referring now also to FIGS. 14A and 14B, the side wall 622 of the guide body 610 is configured to be snap-fit onto and over the hemostasis valve 210 of the second hemostasis valve connector 300. The interior, generally cylindrical, space defined within the side wall 622 is sized to securely and snugly (yet releasably) receive a top portion of the main body 212c of the hemostasis valve 210. The interior of the side wall 622 may include locks, detents, tabs, threads, or other features (not shown) configured to engage mating features provided on the main body 212c, or portions thereof, to more securely engage the hemostasis valve 210 and inhibit the guide body 610 from becoming detached therefrom. When the guide body 610 is positioned over the hemostasis valve as shown in FIG. 14B, the side port 211 of the hemostasis valve 210 is received securely within the cutout 626a, which includes projections 626b that are sized and configured to be snapped around the side port 213 when the side port 213 is positioned within the cutout 626c, thereby securely and snugly attaching the guide body 620 to the hemostasis valve 210. Opening 625 in the top wall 624 is sized and positioned therein to permit access therethrough to the inlet 213 of the hemostasis valve 210.

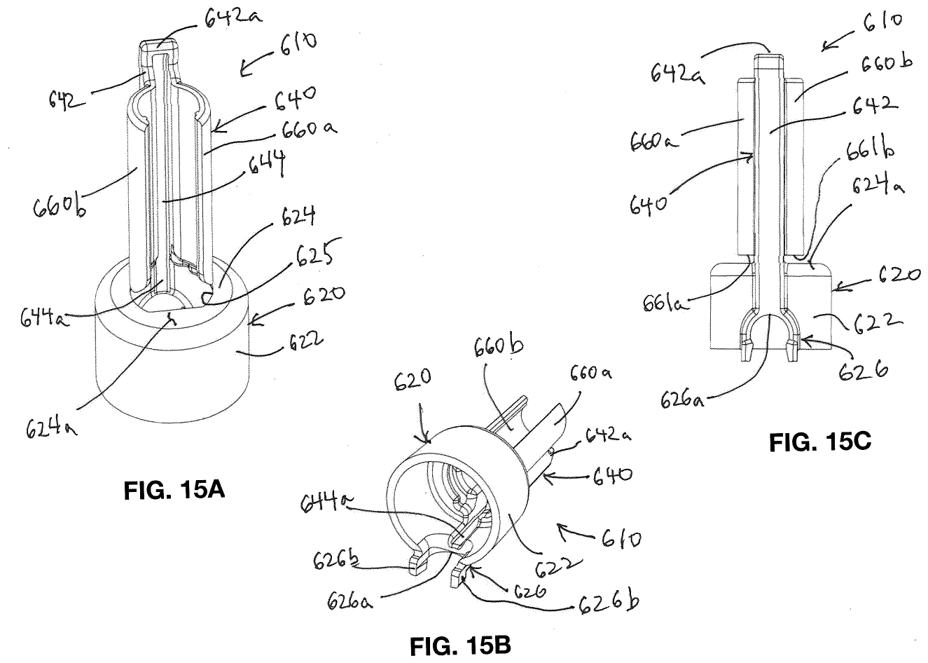

Referring now back to FIGS. 13A-13C, as well as to FIGS. 15A-15C, guide body 610 also includes an upstanding guide post 640 integrally formed with the collar 620 and projecting upwardly from the side wall 622 at a radial location therearound coincident with the cutout 626. Guide post 640 includes a backbone 642 defining an inwardly-facing trough 644 extending upwardly from the top wall 624 to an abutment 642a provided at the proximal end of the backbone 642. A longitudinal slot 644a is provided in the inner wall of the side wall 622 radially coincident with, and open to, the cutout 626a and the through 644, thereby providing a smooth, continuous groove longitudinally from the upwardmost point starting at the abutment and extending downwardly to the cutout 626a.

Guide body 610 further includes first and second arms 660a, 660b, respectively, each extending from, and along, one side wall of the trough 644 and projecting inwardly, generally around and over the opening 526, one on each side thereof. Arms 660a, 660b have a generally outwardly curved-plane, concave shape, thereby generally defining an open tube-like structure suspended over and projecting upwardly from the top wall 624 of the collar 620. Arms 660a, 660b each have a bottom edge 661a, 661b, respectively, that is parallel to, but spaced upwardly from the flat surface 624a of the top wall 624 of the collar 620 by a short distance 670, for purposes that will be described below.

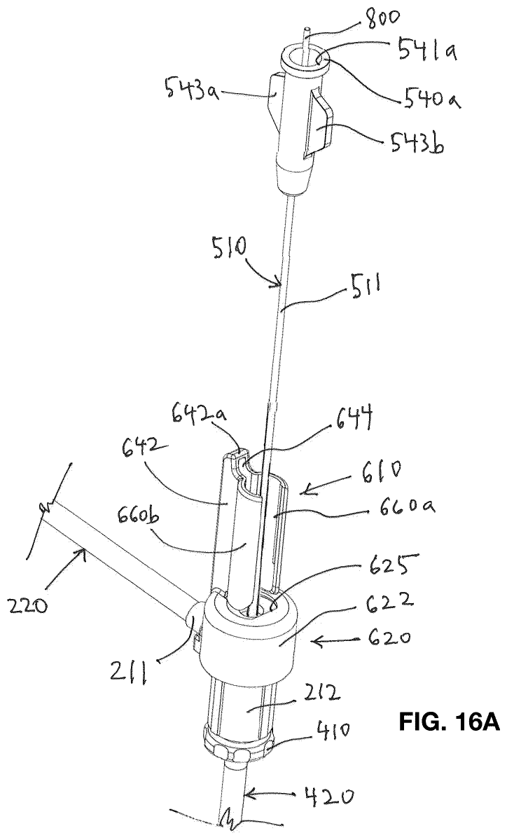

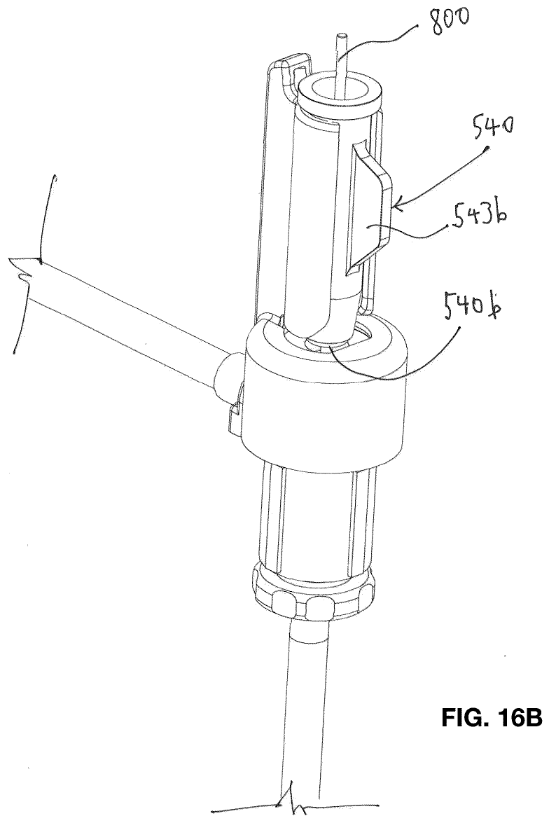

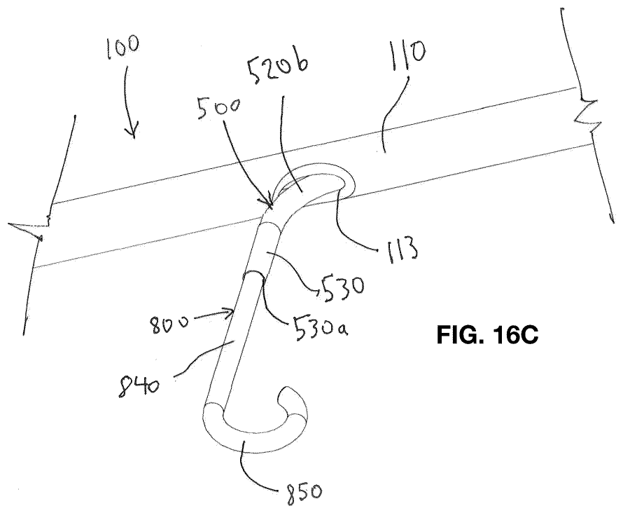

Referring back to the detailed description provided above in reference to FIG. 8, a partially-assembled apparatus 1000 according to an embodiment of the present invention in which the second hemostasis valve connector 300 was shown to be connected to the extension line 400, which was itself connected to the antegrade arm 142 of the retrograde dilator 100, thereby providing a continuous path through which antegrade guidewire 800 (FIG. 16B) could be advanced from the inlet 213 of the hemostasis valve 210 of the second hemostasis valve connector 300 all the way, continuously, to the side port 113 of the retrograde dilator 100. Then, while referring to FIGS. 14A and 14B, a detailed description was provided above in which it was taught that the guide body 610 of the deflection catheter guide 600 may be securely attached to the main body 212c of the hemostasis valve 210 of the second hemostasis valve connector 300.

Now, with reference to FIGS. 16A-16C, a description will be provided in which the antegrade advancement sub-assembly (i.e., the antegrade deflection microcatheter 500 which has been “pre-loaded” with the antegrade guidewire 800) will be loaded into the partially-assembled apparatus 1000 shown in FIG. 8 according to an embodiment of the present invention.

The distal end of the antegrade advancement sub-assembly is inserted into the inlet 213 of the hemostasis valve 210 of the second hemostasis valve connector 300 and advanced through the extension line 400, through the retrograde dilator 100 (via the antegrade arm 142 and the antegrade lumen 112 thereof) and out the side port 113 of the dilator 100. The outside surface of the main body 510 of the microcatheter 500 may be coated with a lubricious agent in order to facilitate smooth advancement through the apparatus 1000. Either way, the fluid pathway through which the antegrade advancement sub-assembly is advanced as just described may be “primed” beforehand, preferably with a bioinert solution such as sterile saline, by connecting a source of such solution (not shown) to a connector port 232a, 232b of the valve 230 of the second hemostasis valve connector 300, opening same and allowing a sufficient quantity of the solution to flush through the fluid pathway in order to activate the lubricious coating.

The length of the main body 510 of the antegrade deflection microcatheter 500 is carefully selected such that when the antegrade advancement sub-assembly is advanced as far distally as possible (i.e., when the distal end 540b of the winged grip 540 is advanced far enough distally to abut the hemostasis valve 210 of the second hemostasis valve connector 300, such as shown in FIG. 16B), the distal extension 530 protrudes from the side port 113 (FIG. 16C) by a small, preselected distance. If the antegrade guidewire 800 is fully deployed as well, as shown in FIG. 16C, the distal extension 840 and the distal hook 850 thereof will protrude even farther from the side port 113 and the open distal end 530b of the microcatheter 500 by a small, preselected distance. Having extended beyond the open distal end 530b of the microcatheter extension 530, the shape of the distal hook 850 is no longer constrained thereby and, under the bias inherent in the material properties of the shape memory material from which it is constructed, the distal hook 850 resumes its inwardly-directed hook orientation, pointing proximally “back” towards the main body 110 of the dilator 100.

The orientation of the microcatheter extension 530, as well as the distal hook 850 of the guidewire 800, is one aspect of the present invention. As will be described in greater detail below with respect to certain methods for providing antegrade advancement of a medical device through a patient's vasculature having first obtained retrograde access thereto, it is preferred that the microcatheter extension 530 and the distal hook 850 of the guidewire 800 is capable of protruding from the side port 113 as shown, for example, in FIG. 2 (i.e., generally lying in the same plane as the various other components of the apparatus 1000). This orientation is also important, for example, to encourage the proper exiting of the antegrade advancement sub-assembly from within the antegrade lumen 112. As the antegrade advancement sub-assembly is being advanced distally through the antegrade lumen 112, the relatively stiff material used to construct the dilator 100 forces the antegrade advancement sub-assembly into a generally straight, or linear, orientation against the bias of the shape memory material from which the antegrade advancement sub-assembly is constructed to “want” to rest in an orientation such as shown in FIG. 11A. If the antegrade advancement sub-assembly were oriented, for example, 180 degrees opposite from what is shown, for example, in FIG. 16C, when advancement of the antegrade advancement sub-assembly through the antegrade lumen 112 causes the distal portion thereof to “meet” the plug 120, the bias of the shape memory material from which the antegrade advancement sub-assembly is constructed would urge the distal portion of the antegrade advancement sub-assembly “away” from the side port 113, not toward (and through) it. As such, according to one aspect of the present invention, the apparatus 1000 according to one embodiment hereof, and more particularly, the microcatheter 500 according to one embodiment hereof, provides a visual cue to the user of the orientation of the antegrade advancement sub-assembly.

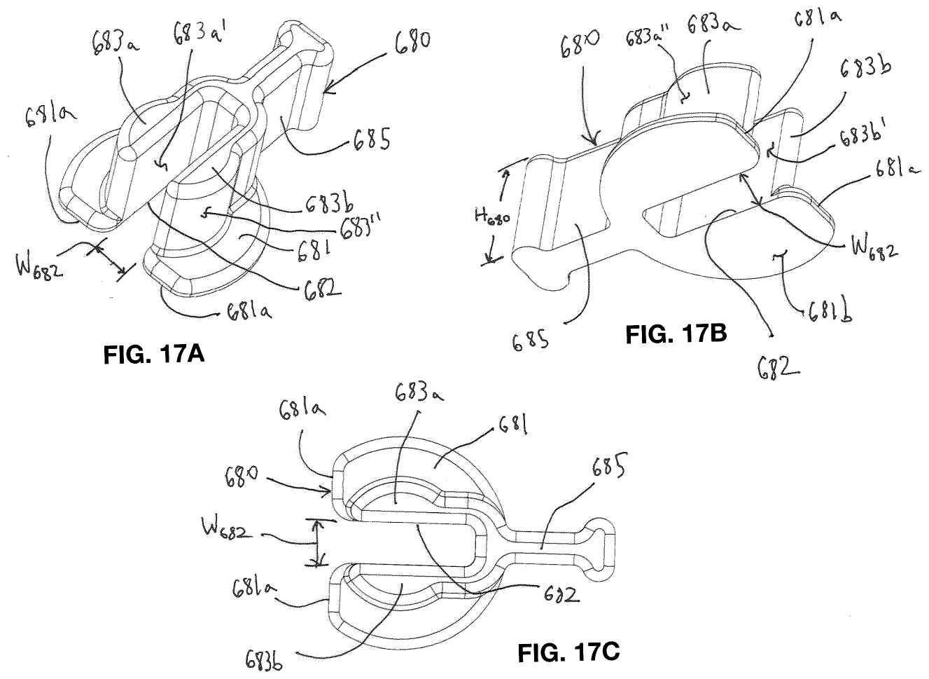

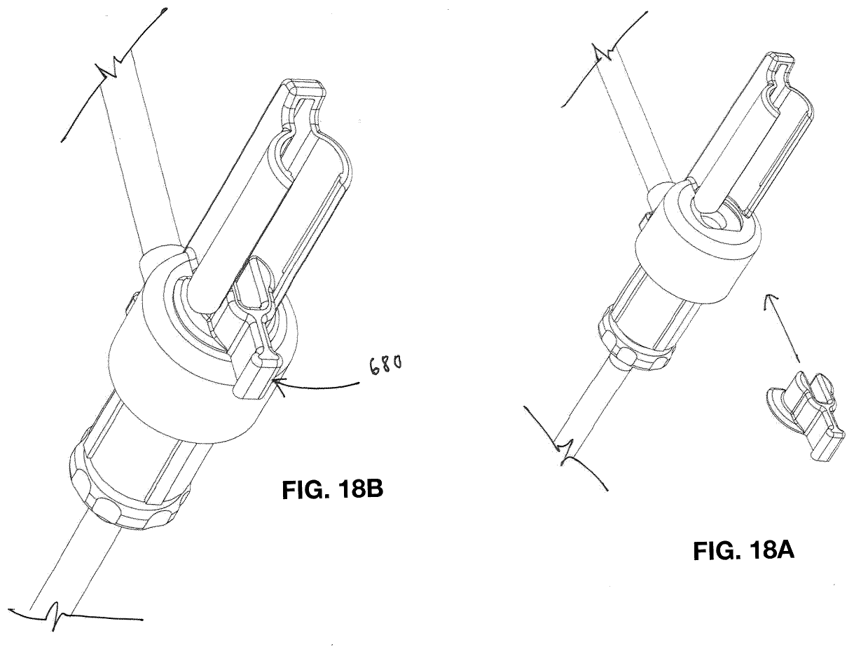

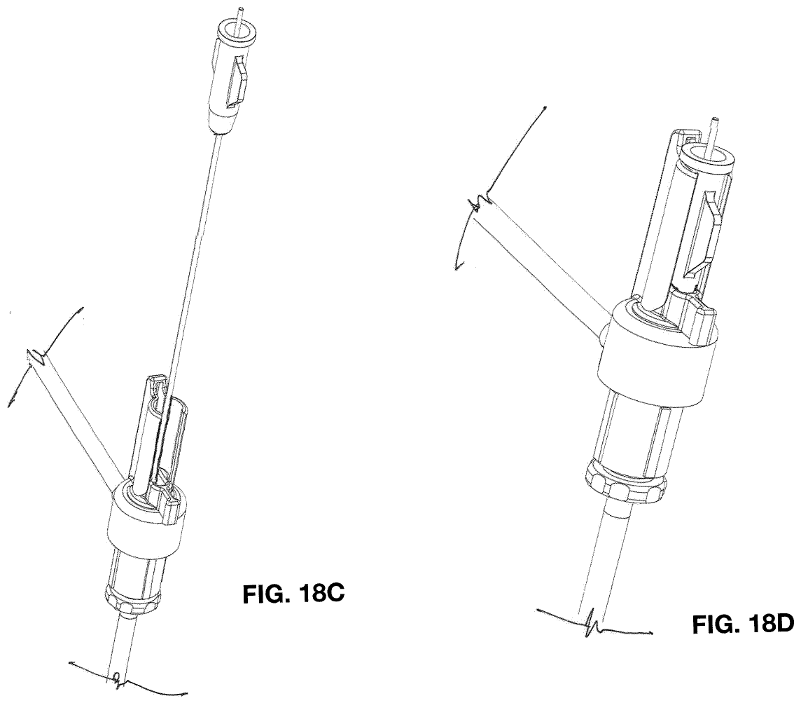

With reference to FIGS. 13A, 15C and 17A-17C, deflection catheter guide 600 (FIG. 18A) includes a clip 680 that is removably insertable into the space formed by the short distance 670 provided between the bottom edges 661a, 661b of the arms 660a, 660b, respectively, and the flat surface 624a of the top wall 624 of the collar 620. Arms 660a, 660b have forward edges 660a′ 660b′ spaced from one another by a distance sufficient to allow clip 680 to pass therebetween. Referring now more specifically to FIGS. 17A-17C, clip 680 includes bottom plane 681 and two opposing, upstanding arms 683a, 683b integrally formed with the bottom plane 681 and from which an upstanding finger grip 685 projects rearwardly. The bottom plane 681 is generally circular and includes a recess 682 projecting rearwardly from a generally flat front edge 681a. Arms 683a, 683b include opposing faces 683a′, 683b′, respectively, spaced from one another by a distance coinciding with a width W₆₈₂ of the recess 682. Opposing faces 683a′, 683b′ of arms 683a, 683b, respectively, cooperate with recess 682 in the bottom plane to define an upstanding channel formed in the front of the clip 680. Bottom plane 681 includes a flat bottom surface 681b.

With reference to FIGS. 18A-18C, the apparatus 1000 according to an embodiment of the present invention is prepared, in part, by assembling certain ones of the components that were described above in a manner as will now be described. First, as shown in FIG. 18A, the guide body 610, and more particularly, the collar 620 of the guide body 610 is affixed, preferably by a snap-fit arrangement, to the hemostasis valve 210 of the second hemostasis valve connector 300, as shown in FIG. 18A.

Next, clip 680 is positioned such that flanking wings of the bottom plane 681 slide into the slots formed between the bottom edges 661a, 661b of the guide body arms 660a, 660b, respectively, and the top wall flat surface 624a. In this configuration, as shown in FIG. 18B, the channel through the clip 680 formed by spaced-apart opposing faces 683a′, 683b′ of arms 683a, 683b, respectively, is axially aligned with the opening 625 in the guide body top wall 624, both of which are aligned with, and open to, the inlet 213 of the hemostasis valve 610. Each arm 683a, 683b of the clip 680 includes a rounded outer surface 683a″, 683b″ facing outwardly opposite its respective opposing channel face 683a′, 683b′. As the clip 680 is being inserted into the main body 610 between first and second arms 660a, 660b, respectively, the forward edges 660a′, 660b′ ride over the leading surfaces of the clip arm outer surfaces 683a″, 683b″ and are forced apart slightly to accommodate the clip 680 therebetween. Main body 610 and the arms 6660a, 660b thereof are made of a sufficiently flexible, and elastic, material to permit the arms 683a, 683b of the clip 680 to pass therebetween. As the clip 680 continues to be inserted into the main body 610, the arms 660a, 660b of the main body 610 will continue to ride over the out surfaces 683a″, 683b″ until they reach the midpoint thereof, at which point, the clip 680 will snap fully into the channel formed between the arms 660a, 660b of the main body 610. At this point, flanking wings of the bottom plane 681 are fully seated within the slots formed between the bottom edges 661a, 661b of the guide body arms 660a, 660b, respectively, and the top wall flat surface 624a, and the clip 680 is removably “locked” in this position.

Referring now to FIGS. 18C and 18D, the antegrade advancement sub-assembly (i.e., the antegrade deflection microcatheter 500 which has been “pre-loaded” with the antegrade guidewire 800) may be fed into the inlet 213 of the hemostasis valve 210 of the second hemostasis valve connector 300, through the extension line 400, and through the dilator 100 until the distal end 540b of the winged grip 540 meets the top surface of the clip arms 683a, 683b. According to one aspect of the present invention, further distal advancement of the antegrade advancement sub-assembly is thereby prevented by the clip 680. As described above, the length of the main body 510 of the antegrade deflection catheter 500 is carefully selected so that when the antegrade advancement sub-assembly is in this position, the open distal end 530 of the microcatheter main body 510 dwells just within the side port 113 (i.e., it does not protrude from the main body 110 of the dilator 100). And while the antegrade guidewire 800 remains moveable within the microcatheter 500 such that it could be advanced distally such that the distal tip 850b thereof protrudes from within the open distal end 530b of the microcatheter main body 510 and outwardly from the side port 213, the antegrade guidewire 800 should rest in a state in which the distal tip 850b thereof dwells within the microcatheter main body 510. A visual indicator (not shown) may be provided near the proximal end thereof to signal to the user that the guidewire 800 should not be advanced distally beyond a certain point to that it is not protruding from the side port 113 of the dilator 100 as the dilator 100 is advanced into a patient's body, as will be described in greater detail below in regard to a description of certain methods for providing antegrade advancement of a medical device through a patient's vasculature having first obtained retrograde access thereto according to certain embodiments of the present invention.

As the user advances the antegrade advancement sub-assembly through the apparatus 1000 and arrives to a point where the distal end 540b of the winged grip 540 approaches the main body 610 of the deflection catheter guide 600, the user orients the antegrade deflection microcatheter 500 such that the first wing 543a is received by, and positioned within, the trough 644 of the main body 610 of the deflection catheter guide 600. This ensures that the second curved section 520a of the microcatheter 500, as well as the distal hook 850 of the antegrade guidewire 800 will be in the proper orientation as they exit the side port 113 as described more fully below. In order to ensure that the user positions the first wing 543a of the winged grip 540 in the trough 644 of the main body 610 of the deflection catheter guide 600, the second wing 543b, which extends opposite the first wing 543a, may be provided with a visual indicator, that may cooperate with another visual indicator provided on the main body 610 of the deflection catheter guide 600.

Clip 680 has a height H₆₈₀ that coincides with a forward, preselected linear distal advancement distance that the antegrade advancement sub-assembly, and more particularly, forward advancement of the open distal end 530b of the microcatheter main body 510, that should be made in order to cause the open distal end 530b

One method according to certain embodiments of the present invention will be described for providing antegrade advancement of a medical device, such as a guidewire, catheter, stent delivery system, percutaneous transluminal angioplasty balloon delivery system, percutaneous transluminal coronary angioplasty balloon delivery system, or the like, through a patient's vasculature into which retrograde access to an artery thereof has been made and through which the medical device is introduced, by an apparatus, such as, for example, the apparatus 1000 shown in FIGS. 1-18D, as well as methods for using an apparatus according to certain embodiments of the present invention, such as the apparatus 1000 shown in FIGS. 1-18D, to achieve antegrade advancement of a medical device while utilizing retrograde access to an artery of the patient's vasculature.

The apparatus 1000 is provided to a healthcare professional pre-assembled as shown and described above with reference, according to one embodiment of the present invention, to FIG. 8 such that the connector 212 of the first hemostasis valve connector 200 is operatively coupled to the retrograde arm 141 of the retrograde dilator Y-connector 140, the male luer connector 430 of the extension line 400 is operatively coupled to the antegrade arm 142 of the retrograde dilator Y-connector 140, and the connector 212 of the second hemostasis valve connector 300 is operatively coupled to the female luer connector 410 of the extension line 400.

As assembled thus, the apparatus 1000 defines a continuous “retrograde” fluid path formed between the first and second ports 232a, 232b of the first hemostasis valve connector 200 and the open distal end 130b of the retrograde dilator 100 via retrograde lumen 111, as well as a separate inlet into the retrograde fluid path by the inlet 213 of the hemostasis valve 210 of the first hemostasis valve connector 200. Similarly, as assembled, the apparatus 1000 also defines a continuous “antegrade” fluid path formed in parallel to the retrograde fluid path between the first and second ports 232a, 232b of the second hemostasis valve connector 300 and the side port 113 of the retrograde dilator 100 via antegrade lumen 112, as well as a separate inlet into the antegrade fluid path by the inlet 213 of the hemostasis valve 210 of the second hemostasis valve connector 300.

With reference now also back to FIG. 1, the apparatus 1000 is further assembled by preloading the retrograde guidewire 700 into the retrograde fluid path of the apparatus 1000 by straightening the curved distal hook 730 and inserting the distal tip 710b thereof into the inlet 213 of the hemostasis valve 210 of the first hemostasis valve connector 200 and feeding the guidewire 700 through the retrograde fluid path until the distal tip 710b protrudes from the open distal end 130b of the retrograde dilator 100. As the retrograde guidewire 700 is fed into the apparatus 1000, the retrograde guidewire 700 straightens out against the bias of any preformed shape such as those shown in FIGS. 9A and 9B, and would return to such preformed shape upon removal from the device 1000. Prior to use, guidewire 700 should be withdrawn partially prior to the procedure such that distal tip 710b thereof does not protrude from the open distal end 130b of the dilator 100, but rather resides therein just proximal to the open distal end 130b, until the appropriate point in the procedure at which it is advanced therefrom as described in greater detail below. Markings (not shown) may be provided near the proximal tip 710a of the guidewire 700 to indicate how far proximally the guidewire 700 should be withdrawn from within the apparatus 1000, pre-procedure, to ensure that the distal tip 710b thereof is not protruding from the open distal end 130b of the dilator 100.

Next, and with reference to FIGS. 14A and 14B, the deflection catheter guide 600 is snap-fit over the inlet 213 of the hemostasis valve 210 of the second hemostasis valve connector 300 and then the clip 680 is snap-fit into the guide body 610 of the catheter guide 600 as described above and shown in FIGS. 18A and 18B.

Now with reference to FIGS. 18C and 18D, antegrade guidewire 800, and more specifically, antegrade advancement sub-assembly (i.e., the antegrade deflection microcatheter 500 and the antegrade guidewire 800), is preloaded into the antegrade fluid path of the apparatus 1000 by straightening the curved distal hook 850 of the guidewire 800, inserting the distal tip 850b thereof into the inlet 213 of the hemostasis valve 210 of the second hemostasis valve connector 300 and feeding the antegrade advancement sub-assembly through the antegrade fluid path until the winged grip 540 of the antegrade deflection catheter 500 meets the clip 680 positioned within the first and second arms 660a, 660b, of the guide body 610. As the antegrade advancement sub-assembly is fed into the apparatus 1000, the antegrade guidewire 800 and the antegrade deflection catheter 500 both straighten out against the bias of any preformed shape such as those shown in FIGS. 10A and 11A, and would return to such preformed shape upon removal from the device 1000.

At the point at which the winged grip 540 of the antegrade advancement sub-assembly approaches the guide body 610 of the deflection catheter guide 600, the antegrade advancement sub-assembly is rotated to align first wing 543a with longitudinal slot 644 of the guide body 610, thereby ensuring that the antegrade advancement sub-assembly, and more particularly, the curved distal hook 850 of the antegrade guidewire 800 is in the proper orientation and remains in the proper orientation during use. A visual indication (not shown) might be provided on either or both the first wing 543a and/or the longitudinal slot 644 to remind the healthcare professional of the proper orientation of the winged grip 540 relative to the guide body 610. Once the winged grip 540 meets the clip 680, further distal advancement of the antegrade advancement sub-assembly is prevented by the clip 680. In this position, the guidewire 800 dwells within the antegrade fluid path such that the distal tip 850b thereof is positioned near, but not projecting from, the side port 113 as shown in FIG. 16C.

The apparatus 1000 is now prepared for providing antegrade advancement of certain medical devices into the vasculature of a patient utilizing retrograde access thereto, as will be described now in even greater detail. The apparatus 1000, thus configured, may be pre-assembled, packaged, sterilized and delivered to a clinical setting, or may be provided as a kit of individual components, each pre-assembled, packaged, sterilized and delivered to a clinical setting for assembly or partial assembly by the healthcare professional responsible for performing the procedure and/or his/her staff.

According to one aspect of the present invention, the various embodiments of the apparatus 1000 and the methods described herein allow a healthcare professional to intervene on an adverse health condition, such as peripheral artery disease including one or more vascular blockages located, for instance, in the lower extremity of a patient's leg. Whereas previous interventional procedures oftentimes required the healthcare professional to gain access to the patient's vasculature via the opposite (i.e., unaffected) leg and advance interventional medical devices “over the horn”, i.e., by gaining retrograde access to the superficial femoral artery in the unaffected leg, thence retrograde through the common iliac artery in the unaffected leg, “over” the iliac bifurcation, thence antegrade through the iliac and superficial femoral arteries of the affected leg to reach the blockage in the affected leg, the present invention allows the healthcare professional to gain access to the patient's vasculature directly via the affected leg. This is accomplished, in part, by utilizing an apparatus, such as the apparatus 1000 described herein as one embodiment of the invention, that provides antegrade advancement of certain conventional medical devices distally within the affected limb but that allows the healthcare professional to gain access to the patient's vasculature directly in the affected limb utilizing retrograde access, which is not only a preferred approach to gaining access to a patient's vasculature, but leads to more favorable results, generally.

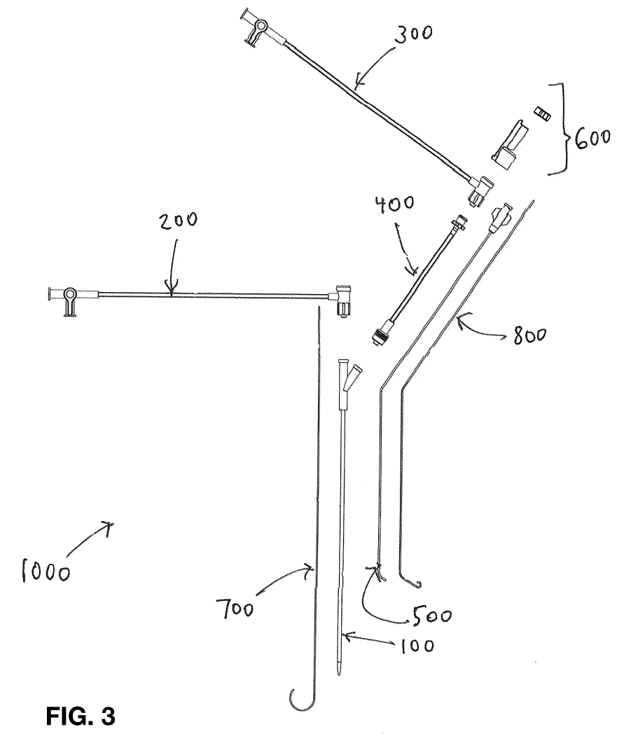

Once a healthcare professional diagnoses that a patient requires such an interventional procedure, the patient is prepared for the procedure utilizing conventional techniques, as well as prepares the clinical setting for the procedure, which includes, among other things, providing the apparatus 1000 prepared and prepackaged in a sterilized kit as described above. To prepare the apparatus 1000 for the procedure, the apparatus 1000 (which is ordinarily provided in a thermoformed tray (not shown) having pockets pre-formed therein that resembles or mimics the apparatus 1000 laying flat on a table, such as shown in FIG. 2). Alternatively, the thermoformed tray may have separate pockets pre-formed to resemble each of the components individually, such as shown, for example, in FIG. 3 to include pockets for each the dilator 100, the first and second hemostasis valve connectors 200, 300, respectively, the extension line 400, the antegrade deflection catheter 500, the deflection catheter guide 600, the retrograde guidewire 700 and the antegrade guidewire 800, as well as other supplies and/or devices that may be necessary or helpful for the procedure.

However the apparatus 1000 is packaged, the outer packaging is removed while the thermoformed tray (in which the apparatus 1000 is provided) is handed across, and into, the sterile field surrounding the patient and received by the healthcare professional standing within the sterile field, all of which has been sterilized and isolated. Once in the sterile field, the apparatus 1000, or its components, is removed from the thermoformed tray and, if not provided in the package in a pre-assembled state, the components are assembled as shown in FIGS. 1 and 2. Next, retrograde guidewire 700 is removed from the retrograde fluid path of the apparatus 1000 and set aside, for instance, in a conventional plastic guidewire bowl (not shown) in which a small pool of sterile saline is provided. Placing the retrograde guidewire 700 in the pool of sterile saline until it is needed may have the effect of activating any lubricious coating that has been applied to the retrograde guidewire 700 during manufacturing/assembly.

Similarly, the antegrade advancement sub-assembly is removed from the antegrade fluid path of the apparatus 1000 and set aside, for instance, in either the same conventional plastic guidewire bowl or another (neither shown) in which a small pool of sterile saline is provided. Placing the antegrade guidewire 800 in the pool of sterile saline until it is needed may have the effect of activating any lubricious coating that has been applied to the antegrade guidewire 800 during manufacturing/assembly.

Next, sterile saline may be flushed through the retrograde fluid path by connecting a source of such saline (not shown) to either first or second ports 232a, 232b, respectively, and applying a pressure thereto to effectuate positive flow of the saline from the source, through the retrograde fluid path and out the open distal end 130b of the dilator 100. Flushing the retrograde fluid path in this manner may have the effect of dislodging any residue or particulate material that may reside within the retrograde fluid path remaining from manufacturing, packaging, etc., activities. Flushing the retrograde fluid path in this manner may also have the effect of activating a lubricious coating that may have been applied to the inner surfaces of the various components defining the retrograde fluid path during manufacturing/assembly. Sterile saline may then be flushed through the antegrade fluid path in a similar manner to achieve similar purposes.

Once the flushing of the antegrade fluid path is complete, the antegrade advancement sub-assembly is loaded into the apparatus 1000 as described in detail above, with the healthcare professional taking special care to ensure that the winged grip 540 is aligned with the longitudinal slot 644 to ensure proper orientation of the curved distal hook 850 of the antegrade guidewire 800 for properly exiting the side port 113 as described below.

The method according to an aspect of the present invention includes the step of gaining retrograde access of the dilator 100 to the superficial femoral artery of a patient's vasculature in an affected leg of the patient in which peripheral artery disease exists and for which performing an interventional procedure is desired. Utilizing ultrasound or another imaging technique, the superficial femoral artery of the patient's affected limb is located and an access point to the superficial femoral artery is chosen slightly distal of the bifurcation between the superficial femoral artery and the femoral profunda. Using an 18-gauge needle, access to the patient's superficial femoral artery is made through the patient's tissue, with an approach, preferably, of about 60 degrees retrograde relative to the direction of what would be normal, healthy blood flow through the patient's superficial femoral artery toward the patient's feet (i.e., near where the intervention is desired). In other words, the healthcare provider gains access to the patient's vasculature directly in the affected limb (rather than the opposite limb, as is the conventional technique) using an approach that is directed, generally, toward the patient's head (i.e., “away from” the distal location in the patient's limb near where the intervention is desired).

Once a distal end of the needle has pierced the superficial femoral artery, thereby gaining access thereto, the retrograde orientation of the needle at 60 degrees is maintained while the retrograde guidewire 700 is advanced into the patient's vasculature in a retrograde direction via the needle. More specifically, the curved distal hook 730 of the retrograde guidewire 700 is straightened and the distal tip 710b thereof is fed into an open proximal end of the needle and advanced distally until the distal tip 710b of the guidewire 700 protrudes from the distal tip of the needle into the artery. The healthcare professional then manipulates the guidewire 700 by rotating it back-and-forth while observing it under ultrasound to ensure that as the guidewire 700 is further advanced distally into the artery, the direction of the curved bias (which the curved distal hook 730 of the guidewire will resume as it exits the distal tip of the needle) advances in the retrograde direction (i.e., toward the patient's head) into the artery. To accomplish this, the healthcare professional observes, under ultrasound, the orientation of the curved distal hook 730 of the guidewire 710 as it is advanced distally from the distal tip of the needle and rotates the guidewire 700 simultaneously while advancing and withdrawing the guidewire 700 in small increments. Once the healthcare professional determines the proper orientation and placement of the curved distal hook 730 within the artery, the guidewire 700 is advanced further into the artery, if necessary, until a sufficient length thereof dwells within the artery to provide proper anchoring thereof. The needle is then removed from the patient's body, leaving the guidewire 700 in place over which it provides a path through the patient's tissue and into the patient's superficial femoral artery of the affected limb and toward the patient's head (i.e., away from the treatment site in the lower extremity of the patient's affected limb).

Next, a method of gaining retrograde access to the superficial femoral artery according to one embodiment of the present invention further includes the step of advancing the retrograde dilator 100 of the apparatus 1000 over the guidewire 700 until the open distal end 130b of the dilator 100 is positioned within the artery. To accomplish this, the healthcare professional inserts the proximal tip 710a of the guidewire 700 into the distal opening of the tip lumen 131 (which, as described above, communicates with the retrograde lumen 111 of the dilator main body 110) and advances the dilator 100 along the guidewire 700 while holding the guidewire 700 in position relative to the patient's body so that it is not inadvertently withdrawn therefrom.

As the distal tip 132 of the dilator 100 arrives to the surface of the patient's body, the healthcare professional gently advances the dilator 100 into the patient's body, through the patient's tissue, along the path toward the superficial femoral artery defined by the in-dwelling guidewire 700 at an angle of about 60 degrees retrograde. As the open distal end 130b of the dilator 100 enters the artery, a small amount of blood may flash back through the retrograde fluid path, around the guidewire 700 positioned therein, and bypass through the transparent flashback tube 200 of the first hemostasis valve connector 200, thereby providing a visual indication to the healthcare professional that the distal tip 132 of the dilator 100 has entered the artery.

The dilator 100 is further advanced into the artery, in a retrograde direction defined by the orientation of the guidewire 700, until the side port 113 passes into the artery. This may indicated visually to the healthcare professional by observing a small amount of blood flashing back through the antegrade fluid path, around the antegrade advancement sub-assembly, and bypass through the transparent flashback tube 220 of the second hemostasis valve connector 300. As the dilator 100 is advanced over the guidewire 700 and into the artery as described above, the proximal tip 710a of the guidewire 700 passes proximally through the continuous retrograde fluid path formed by the retrograde lumen 111 of the dilator 100, the retrograde channel 145 of the dilator Y-connector 140 and the connector 212 of the first hemostasis valve connector 200, until the proximal tip 710a protrudes proximally from the hemostasis valve 210 of the first hemostasis valve connector 200 and is observed by the healthcare professional as a visual indication (along with the ultrasound display) that the distal end of the dilator 100 has achieved access to the artery. A colored stripe or other visual indicia may be provided near the proximal end of the guidewire 700 to provide the healthcare professional with a visual indication of the position/orientation of the guidewire 700 relative to the dilator 100.

Proper positioning and orientation of the side port 113 within the artery (as will be described in greater detail below) enhances the effectiveness of the methods described herein. The side port 113 and the antegrade deflection catheter 500 (and more particularly, the second curved section 520b thereof) cooperate to promote the proper antegrade orientation of the antegrade guidewire 800 as it is advanced distally to project from the side port 113 in an antegrade direction relative to the patient's vasculature, for purposes that are described in greater detail elsewhere herein. To promote proper orientation thereof, the healthcare professional carefully advances and withdraws the dilator 100, while at the same time also rotating it, and observes the degree to which blood flashes back into the flashback tube 220 (and also, alternatively, either the first or second ports 232a, 232b, respectively, of the valve 230 of the second hemostasis valve connector 300, which may be opened to allow for blood to flow therefrom as a further visual indication that the side port 113 is positioned within the artery). While maintaining a 60 degree retrograde orientation of the dilator 100, and with reference to both ultrasound imaging and the flashback indication, the dilator 100 is advanced into the artery until the side port 113 is just within the arterial wall and then rotated until the side port 113 points, generally, in the antegrade direction. The healthcare professional may make several attempts at this, utilizing both ultrasound visualization as well as flashback visualization, to know when the side port 113 is oriented optimally to provide proper antegrade advancement of the medical devices as described in greater detail herein.

Once the healthcare professional is content that the side port 113 is in the proper position and orientation (i.e., the side port 113 is located within the artery immediately distal of the proximal arterial wall and pointed in the antegrade direction), the healthcare professional removes the clip 680 from the guide body 610, thereby permitting distal advancement of the antegrade advancement subassembly by a distance (equal to the ⅜″ of the clip 680) sufficient for the distal end of the antegrade deflection microcatheter 500 to project from the side port 113 into the artery curving in the antegrade direction under the curved bias of the shape memory material used to form the second curved section 520b of the antegrade deflection microcatheter 500. In addition, proximal end 120a of antegrade lumen plug 120 may be formed with a curved, angled or beveled “ramp” shape to facilitate a smooth transition of the distal end of the antegrade deflection microcatheter 500 and the antegrade guidewire 800, although this may not be necessary due to the curved bias of the shape memory material used to form both the antegrade deflection microcatheter 500 and the antegrade guidewire 800. So long as the antegrade deflection microcatheter 500 and the antegrade guidewire 800 are loaded into the dilator 100 at the proper orientation, then the distal ends thereof will be biased towards the side port 113 as the antegrade advancement assembly is advanced distally through the antegrade lumen 112, and will exit the side port 113 in the proper direction/orientation.

After the healthcare professional confirms via ultrasound imaging that the distal end of the antegrade deflection microcatheter 500 and the antegrade guidewire 800 positioned therein are projecting from the side port 113 and are oriented in a proper antegrade direction, the healthcare professional then advances the antegrade guidewire 800 distally from the proximal end thereof, thereby advancing the distal end in the antegrade direction, a sufficient distance to “anchor” the guidewire 800 within the artery so as to facilitate advancement of certain medical devices thereover (as will be described in greater detail below). As the guidewire 800 is being deployed from the dilator 100, and more specifically, from the antegrade deflection catheter 500 positioned therein except for the distal end thereof, which protrudes a small curved distance in the antegrade direction therefrom, the healthcare professional manipulates the apparatus 1000 and the antegrade guidewire 800 under ultrasonic imaging small distances toward and away from the artery, while at the same time rotating the apparatus 1000, observing the advancement of there distal end of the guidewire 800 to ensure proper antegrade direction and orientation thereof. The indwelling presence of the retrograde guidewire 700 in the retrograde direction dwelling within the artery acts to anchor the apparatus 1000 in a generally retrograde angular orientation so as to facilitate proper antegrade advancement of the antegrade guidewire 800 in the opposite direction.

Once the healthcare professional is comfortable that the antegrade guidewire 800 has advanced in the antegrade direction in the artery a sufficient distance to be anchored stably therein, the retrograde guidewire 700 is first removed from the patient by slowing withdrawing the proximal end thereof from the apparatus 1000. Next, the antegrade deflection microcatheter 500 is carefully removed from the patient, while holding the antegrade guidewire 800 firmly in place so as to prevent its inadvertent withdrawal or removal from the artery. Once the antegrade deflection catheter 500 has been removed from the patient, the dilator 100 is removed from the patient over the antegrade guidewire 800 while holding the antegrade guidewire 800 firmly in place so as to prevent its inadvertent withdrawal or removal from the patient or from the artery.

Once all but the antegrade guidewire 800 has been removed from the patient's body, conventional access products, such as an access dilator, sheath, catheter, or the like, can be introduced into the patient's vasculature of the affected limb, over the antegrade guidewire 800 in the antegrade direction, and thereafter, certain diagnostic or therapeutic interventional medical devices, such as, for example, a stent delivery system, a percutaneous transluminal angioplasty balloon delivery system, a percutaneous transluminal coronary angioplasty balloon delivery system, or the like, may be introduced into the patient's vasculature in the antegrade direction in order to perform an interventional procedure according to conventional techniques.

While exemplary methods and compositions have been illustrated by describing examples, and while the examples have been described in considerable detail, it is not the intention of the applicant to restrict or in any way limit the scope of the appended claims to such detail. It is, of course, not possible to describe every conceivable combination of components or methodologies for purposes of describing the systems, methods, devices, and so on, described herein. Additional advantages and modifications will readily appear to those skilled in the art. Therefore, the invention in not limited to the specific details, the representative revascularization catheter system, and the illustrative examples shown and described. Thus, this application is intended to embrace alterations, modifications, and variations that fall within the scope of the appended claims. Furthermore, the preceding description is not meant to limit the scope of the invention. Rather, the scope of the invention is to be determined by the appended claims and their equivalents.

For instance, under certain circumstances, performance and efficacy of the apparatus 1000 might be improved if the antegrade guidewire 800 (and the microcatheter 500 that assists in positioning it within the patient's vasculature as described in greater detail above) is introduced via the first hemostasis valve 210 of the first hemostasis valve connector 200 rather than via the first hemostasis valve 210 of the second hemostasis valve connector 300, such as is described above according to one embodiment hereof shown in FIG. 8, as an example. Such a modified arrangement may be preferred, for example, because it permits both the microcatheter 500 and the antegrade guidewire 800 passing therethrough to be introduced via the “straight” retrograde arm 141 of the Y-connector 140 rather than the “angled” antegrade arm 142 of the Y-connector 140, thereby improving “pushability” of both the microcatheter 500 and the antegrade guidewire 800 through dual lumen dilator 100 and distally out of, and in a partially-reversed direction from, the side port 113 thereof.

In order for such a modified arrangement to provide the outcomes described herein, several additional modifications might need to be made to the apparatus 1000, either prior to, or during, use thereof by a healthcare professional. For example, if the first hemostasis valve 210 of the first hemostasis valve connector 200 is intended to be used to introduce the microcatheter 500 and the antegrade guidewire 800 into the patient's vasculature rather than the first hemostasis valve 210 of the second hemostasis valve connector 300, as described above, then the first hemostasis valve 210 of the second hemostasis valve connector 300 should be used, rather than the first hemostasis valve 210 of the first hemostasis valve connector 200, to introduce the retrograde guidewire 700 into the patient's vasculature. However, since, as described above with respect to certain embodiments hereof, side port 113 is in communication with antegrade lumen 112 of the dilator 110, which is itself in communication with the antegrade arm 142 of the Y-connector 140 (and thus, the first hemostasis valve 210 of the second hemostasis valve connector 300), not the retrograde lumen 111 of the dilator 110 (and thus, with neither the retrograde arm 141 of the Y-connector 140 nor the first hemostasis valve 210 of the first hemostasis valve connector 200, the antegrade lumen 112 and the retrograde lumen 111 of the dilator 110 must be “flipped” such that side port 113 communicates with retrograde arm 141 of the Y-connector 140 and that open distal tip 130b of the dilator communicates with antegrade arm 142 of the Y-connector 140. This can be accomplished, in one exemplary way, by simply rotating the dual lumen dilator 110 around its longitudinal axis by 180 degrees before affixing it to the Y-connector 140. Other arrangements for establishing proper communication between the ports of the various components to achieve the functionality described herein are envisioned to fall within the spirit and the scope of the present invention.

In addition, deflection catheter guide 600 should be attached, as described above, to the first hemostasis valve 210 of the first hemostasis valve connector 200 rather than to the first hemostasis valve 210 of the second hemostasis valve connector 300.

These, and other, modifications will be apparent to those of ordinary skill in the art upon reading the within disclosure.

What is claimed is:

1. An apparatus for providing access to a patient's vasculature, comprising:
a dilator having a distal tip, a proximal end and a main body extending therebetween, the dilator having a first lumen extending from the proximal end to the distal tip and a second lumen extending in parallel to the first lumen between the proximal end and a side port provided in the main body spaced proximal to the distal tip by a distance;
a first hemostasis valve in fluid communication with the first lumen at the proximal end of the dilator;
a second hemostasis valve in fluid communication with the second lumen at the proximal end of the dilator;
a retrograde guidewire positionable within the first lumen for advancement therein from the proximal end toward the distal tip and adapted to project therefrom by a first preselected distance;
a deflection microcatheter positionable within the second lumen for advancement therein from the proximal end toward the side port and adapted to project therefrom by a second preselected distance; and,
an antegrade guidewire positionable within a lumen of the deflection microcatheter for advancement therein from the proximal end toward the side port and adapted to project therefrom by a third preselected distance.

2. The apparatus of claim 1, wherein the first and second hemostasis valves are connected to the proximal end of the dilator by a Y-connector such that the first hemostasis valve is in fluid communication with the first lumen of the dilator and the second hemostasis valve is in fluid communication with the second lumen of the dilator.

3. The apparatus of claim 2, wherein the first hemostasis valve is connected to a retrograde arm of the Y-connector and wherein the second hemostasis valve is connected to an antegrade arm of the Y-connector.

4. The apparatus of claim 3, wherein the first hemostasis valve is connected to the retrograde arm of the Y-connector by a first hemostasis valve connector and wherein the second hemostasis valve is connected to the antegrade arm of the Y-connector by a second hemostasis valve connector.

5. The apparatus of claim 3, wherein the first hemostasis valve is connected to the retrograde arm of the Y-connector by a first hemostasis valve connector, wherein the second hemostasis valve is connected to a proximal end of an extension line by a second hemostasis valve connector and wherein a distal end of the extension line is connected to the antegrade arm of the Y-connector.

6. The apparatus of claim 3, wherein the first hemostasis valve is connected to the retrograde arm of the Y-connector by a first hemostasis valve connector and wherein the first hemostasis valve connector comprises a flashback tube.

7. The apparatus of claim 3, wherein the second hemostasis valve is connected to the retrograde arm of the Y-connector by a second hemostasis valve connector and wherein the second hemostasis valve connector comprises a flashback tube.

8. The apparatus of claim 1, further comprising a plug disposed within the antegrade lumen between the side port and the distal tip.

9. The apparatus of claim 1, wherein the retrograde guidewire comprises a curved distal hook section.

10. The apparatus of claim 1, wherein the antegrade guidewire comprises a curved distal hook section.

11. The apparatus of claim 1, wherein the deflection microcatheter comprises a curved distal section.

12. The apparatus of claim 1, further comprising a deflection catheter guide attached to the second hemostasis valve.

13. The apparatus of claim 12, wherein a proximal end of the deflection microcatheter includes a winged grip adapted to engage a portion of the deflection catheter guide, whereby a rotational orientation of the deflection microcatheter is controllably selected by engaging the winged grip with the portion of the deflection catheter guide.

14. The apparatus of claim 12, further comprising a removable clip adapted to engage the deflection catheter guide, whereby the clip prevents distal advancement of the deflection microcatheter when the clip is engaged with the deflection catheter guide.

15. The apparatus of claim 14, wherein the clip includes a height that coincides with the second preselected distance by which the deflection microcatheter is advanced to cause a distal end thereof to project from the side port.

16. The apparatus of claim 1, wherein a distal end of the deflection microcatheter is adapted to project from the side port in a lateral direction.
